(19) United States
(12) Patent Application Publication
(10) Pub. No.: US 2005/0090899 A1
(43) Pub. Date: Apr. 28, 2005

(54) Methods and apparatuses for treating the spine through an access device

(76) Inventor: Gene DiPoto, Upton, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/693,250

(22) Filed: Oct. 24, 2003

(51) Int. Cl. A61F 2/02; A61M 29/00

(52) U.S. Cl. 623/17.11; 623/16.11; 623/11.11

(57) ABSTRACT

In treating the spine of a patient, an access device is inserted through a minimally invasive incision in the skin of the patient, and advanced until a distal portion of the access device is located adjacent the spine. The access device is expanded from a first configuration to a second configuration, the second configuration having an enlarged cross-sectional area at the distal portion thereof such that the distal portion extends across at least a portion of an intervertebral space. A replacement disc nucleus is delivered through the access device into the intervertebral space.

### BACKGROUND OF THE INVENTION

1. Field of the Invention

This application generally relates to methods and apparatuses for performing minimally invasive surgery, and more particularly to methods and apparatuses for performing procedures on a spinal disc of a patient.

2. Description of the Related Art

In the past, patients suffering from degenerative spine conditions, such as progressive degeneration of intervertebral discs, have been treated through open spine surgery. Open spine surgery can provide benefits for such patients. However, such surgery often causes additional trauma, which can itself be very painful. Open surgery can cause a great deal of trauma because the surgeon typically makes large incisions and cuts or strips muscle tissue surrounding the spine to provide open access to the troubled area. In addition, nerve tissue in the area is exposed, and therefore is at risk to injury. Consequently, open surgical procedures carry significant risks of scarring, pain, and blood loss and subject patients to extended recovery times.

Less invasive techniques have been proposed to reduce the trauma of open spine surgery. Such techniques generally reduce the size of the incision and the degree of muscle stripping in order to access the vertebrae. A constant diameter cannula is one apparatus that has been proposed to reduce incision size. The constant diameter cannula is made narrow in order to provide a small entry profile. Unfortunately, the cannula provides minimal space for the physician to observe the body structures and manipulate surgical instruments because it is so narrow.

Fixation and fusion are two procedures that are sometimes performed in combination to reduce the pain associated with degeneration of the intervertebral discs. Fusion involves the replacement of an intervertebral disc with a bone graft intended to fuse the adjacent vertebrae together. Fixation provide an external structure that bridges from one vertebra to an adjacent vertebra to eliminate motion therebetween. While fusion and fixation may reduce some symptoms of spinal degeneration, the long-term health of the spine would be better preserved if some degree of motion could be preserved between the vertebrae on either side of the degraded disc.

### SUMMARY OF THE INVENTION

Accordingly there is a need in the art for minimally invasive apparatuses and methods for treating an intervetebral disc, e.g., the nucleus pulposus, in a manner that maintains or improves motion of vertebrae on either side of the disc. These apparatuses and methods could restore much of the biomechanical functionality of a healthy disc, and provide support and flexibility to adjacent vertebrae in a manner approximating that of a natural nucleus pulposus.

In one embodiment, a portion of a disc of a patient is replaced. The disc has an annulus and a nucleus. An access device is inserted through an incision in the skin of the patient generally postero-laterally. The access device is advanced until a distal portion thereof is located adjacent the spine. The access device is inserted in a first configuration that has a first cross-sectional area at the distal portion thereof. The access device is configured such that the distal portion thereof is enlarged from the first configuration to a second configuration. In the second configuration, the distal portion extends across at least a portion of the disc. An implement is advanced through the access device to the intervertebral space. An aperture is formed in the annulus. A disc evacuation tool is advanced through the access device and through the aperture. At least a portion of the nucleus is removed through the access device to at least partially evacuate the intervertebral space. A replacement disc nucleus is delivered into the partially evacuated intervertebral space through the access device.

In another embodiment, the spine of a patient is treated. An access device is inserted through a minimally invasive incision in the skin of the patient. The access device is advanced until a distal portion thereof is located adjacent the spine. The access device is expanded from a first configuration to a second configuration. The second configuration of the access device has an enlarged cross-sectional area at the distal portion thereof such that the distal portion extends across at least a portion of a disc. A replacement disc nucleus is delivered into the intervertebral space through the access device.

In another embodiment, a device is used to provide access to a surgical location within a patient. The device has an elongate body having a proximal end, a distal end, and a passage extending therebetween. The elongate body defines a length between the proximal and distal ends, such that the proximal end can be positioned outside the patient and the distal end can be positioned inside the patient adjacent the surgical location. The distal end is shaped to the contours of the surgical location. The elongate body is actuatable between a first configuration sized for insertion into the patient and a second configuration, wherein the cross-sectional area of said passage at a first location is greater than the cross-sectional area of said passage at a second location, wherein the first location is distal to the second location.

In another embodiment, a device provides access to a surgical location within a patient.. The device includes an elongate body that has a proximal end, a distal end, and a passage extending therebetween. The elongate body defines a length between the proximal and distal ends such that the proximal end can be positioned outside the patient and the distal end can be positioned inside the patient adjacent- the surgical location. The distal end is shaped to substantially conform to a contour of an anatomical structure near the surgical location. The elongate body is actuatable between a first configuration sized for insertion into the patient and a second configuration wherein the cross-sectional area of the passage at a first location is greater than the cross-sectional area of the passage at a second location, wherein the first location is distal to the second location.

In another embodiment, a device for accessing an intervertebral disc of a patient having a nucleus and an annulus has an elongate body. The elongate body has a proximal end, a distal end, and a passage extending therebetween. The elongate body defines a length between the proximal and distal ends such that the proximal end can be positioned outside the patient and the distal end can be advanced inside the patient and into the annulus. The elongate body is actuatable between a first configuration sized for advancement into the annulus and a second configuration wherein the cross-sectional area of the passage at a first location is greater than the cross-sectional area of the passage at a second location, wherein the first location is distal to the second location.

In another embodiment, a device for accessing an intervertebral disc of a patient having a nucleus and an annulus is provided. The device includes an elongate body and a viewing element. The elongate body has a proximal end, a distal end, a passage extending therebetween, and a viewing element aperture. The viewing element aperture is located near the distal end. The elongate body defines a length between the proximal and distal ends such that when the distal end is advanced into the patient to the annulus, the proximal end is positioned outside the patient. The viewing element extends through the aperture into the passage.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a perspective view of one embodiment of a surgical system and one embodiment of a method for treating the spine of a patient;

FIG. 2 is a perspective view of one embodiment of an expandable conduit in a reduced profile configuration;

FIG. 3 is a perspective view of the expandable conduit of FIG. 2 in a first enlarged configuration;

FIG. 4 is a perspective view of the expandable conduit of FIG. 2 in a second enlarged configuration;

FIG. 5 is a view of one embodiment of a skirt portion of an expandable conduit;

FIG. 6 is a view of another embodiment of a skirt portion of an expandable conduit;

FIG. 7 is a perspective view of another embodiment of an expandable conduit in an enlarged configuration;

FIG. 8 is an enlarged sectional view of the expandable conduit of FIG. 7 taken along lines 8-8 of FIG. 7;

FIG. 9 is a sectional view of the expandable conduit of FIG. 7 taken along lines 9-9 of FIG. 7;

FIG. 10 is a perspective view of another embodiment of an expandable conduit in an enlarged configuration;

FIG. 11 is an enlarged sectional view of the expandable conduit of FIG. 10 taken along lines 11-11 of FIG. 10;

FIG. 12 is a sectional view of the expandable conduit of FIG. 10 taken along lines 12-12 of FIG. 10;

FIG. 13 is a view of a portion of another embodiment of the expandable conduit;

FIG. 14 is a view of a portion of another embodiment of the expandable conduit;

FIG. 15 is a sectional view illustrating one embodiment of a stage of one embodiment of a method for treating the spine of a patient;

FIG. 16 is a side view of one embodiment of an expander apparatus in a reduced profile configuration;

FIG. 17 is a side view of the expander apparatus of FIG. 16 in an expanded configuration;

FIG. 18 is a sectional view of the expander apparatus of FIGS. 16-17 inserted into the expandable conduit of FIG. 2, which has been inserted into a patient;

FIG. 19 is a sectional view of the expander apparatus of FIGS. 16-17 inserted into the expandable conduit of FIG. 2 and expanded to the expanded configuration to retract tissue;

FIG. 20 is an exploded perspective view of one embodiment of an endoscope mount platform;

FIG. 21 is a top view of the endoscope mount platform of FIG. 20 coupled with one embodiment of an indexing arm and one embodiment of an endoscope;

FIG. 22 is a side view of the endoscope mount platform of FIG. 20 illustrated with one embodiment of an indexing arm and one embodiment of an endoscope;

FIG. 23 is a perspective view of one embodiment of an indexing collar of the endoscope mount platform FIG. 20;

FIG. 24 is a perspective view of one embodiment of an endoscope;

FIG. 25 is a partial sectional view of one embodiment of a stage of one embodiment of a method for treating the spine of a patient;

FIG. 26 is a perspective view of one embodiment of a fastener;

FIG. 27 is an exploded perspective view of the fastener of FIG. 26;

FIG. 27(a) is an enlarged side view of one embodiment of a biasing member illustrated in FIG. 27 taken from the perspective of the arrow 27a;

FIG. 28 is a perspective view of one embodiment of a surgical instrument;.

FIG. 29 is an enlarged sectional view of the fastener of FIGS. 26-27 coupled with the surgical instrument,of FIG. 28, illustrating one embodiment of a stage of one embodiment of a method for treating the spine of a patient;

FIG. 30 is side view of one embodiment of another surgical instrument;

FIG. 31 is a partial sectional view of one embodiment of a stage of one embodiment of a method for treating the spine of a patient;

FIG. 32 is a side view of one embodiment of another surgical instrument;

FIG. 33 is a perspective view similar to FIG. 31 illustrating the apparatuses of FIGS. 26 and 32, in one embodiment of a stage of one embodiment of a method for treating the spine of a patient;

FIG. 34 is an enlarged sectional view of the apparatus of FIGS. 26 and 32, illustrating one embodiment of a stage of one embodiment of a method for treating the spine of a patient;

FIG. 35 is an enlarged sectional similar to FIG. 34, illustrating one embodiment of a stage of one embodiment of a method for treating the spine of a patient;

FIG. 36 is an enlarged view in partial section illustrating one embodiment of a stage of one embodiment of a method for treating the spine of a patient;

FIG. 37 is a partial view illustrating one embodiment of a stage of one embodiment of a method for treating the spine of a patient;

FIG. 38 is a perspective view of a first embodiment of a spinal implant showing a first side surface thereof;

FIG. 39 is a perspective view of the spinal implant of FIG. 38 showing a second side surface thereof;

FIG. 40 is a plan view of the spinal implant of FIG. 38 showing an upper surface thereof;

FIG. 41 is a side view of the spinal implant of FIG. 38 showing the first side surface thereof;

FIG. 42 is a cross-sectional view of the spinal implant of FIG. 38 taken along the line 42-42 in FIG. 41;

FIG. 43 is a perspective view of another embodiment of a spinal implant showing a first side surface thereof;

FIG. 44 is a perspective view of the spinal implant of FIG. 43 showing a second side surface thereof;

FIG. 45 is a plan view of the spinal implant of FIG. 43 showing an upper surface thereof;

FIG. 46 is a side view of the spinal implant of FIG. 43 showing the first side surface thereof;

FIG. 47 is a cross-sectional view of the spinal implant taken along the line 47-47 in FIG. 46;

FIG. 48 is a view showing a pair of the spinal implants of FIG. 38 in first relative positions between adjacent vertebrae;

FIG. 49 is a view showing a pair of the spinal implants of FIG. 38 in second relative positions between adjacent vertebrae;

FIG. 50 is a view showing the spinal implant of FIG. 43 between adjacent vertebrae; and

FIG. 51 is a view showing one embodiment of a procedure whereby a spinal implant is inserted between the adjacent vertebrae;

FIG. 52 is a side view of another apparatus that can be employed in a spinal procedure;

FIG. 53 is a front view of the apparatus of FIG. 52;

FIG. 54 is a top view of the apparatus of FIG. 52;

FIG. 55 is a back view of the apparatus of FIG. 52;

FIG. 56 is a bottom view of the apparatus of FIG. 52;

FIG. 57 is a sectional view of an system including the apparatus of FIG. 52 and an access device, which assembly has been inserted within a patient;

FIG. 58 is a longitudinal sectional view of a proximal section of the system of FIG. 57 taken from line 58-58 of FIG. 57;

FIG. 59 is a transverse sectional view of the system of FIG. 58 taken from line 59-59 of FIG. 58;

FIG. 60 is a sectional view, similar to FIG. 57, illustrating an alternative position of the apparatus of FIG. 52;

FIG. 61 is a sectional view, similar to FIG. 57, illustrating another alternative position of the apparatus of FIG. 52;

FIG. 61a is a transverse sectional view of the system of FIG. 61, taken along lines 61a-61a of FIG. 61;

FIG. 62 is a side view, similar to FIG. 52, of another apparatus that can be employed in a surgical procedure;

FIG. 63 is a front view, similar to FIG. 55, of the embodiment of FIG. 62;

FIG. 64 is a sectional view, similar to FIG. 57, of the apparatus of FIGS. 62-63, incorporated into a system which has been inserted into a patient;

FIG. 65 is a transverse sectional view of the apparatus of FIGS. 62-63, taken along lines 65-65 of FIG. 64;

FIG. 66 is a perspective view of a replacement disc nucleus comprising a compliant enclosure;

FIG. 67A is a perspective view of a replacement disc nucleus that incorporates a hydrogel;

FIG. 67B is a side, sectional view of the replacement spinal disc nucleus of FIG. 67A along the line 67B-67B;

FIG. 67C is a top, sectional view of the replacement spinal disc nucleus of FIG. 67A along the line 67C-67C;

FIG. 68 is a perspective view of the replacement spinal disc nucleus of FIG. 67A in a hydrated state;

FIG. 69 is a schematic diagram of a spine of a patient with one embodiment of a replacement disc nucleus implanted therein;

FIG. 70 is a plan view of the replacement disc nucleus of FIG. 69;

FIG. 71 is a diagram representing the spine of a patient with another embodiment of a replacement disc nucleus implanted therein;

FIG. 72 is a perspective view illustrating one embodiment of a replacement disc nucleus;

FIG. 73 is a schematic view of one surface of a vertebra that defines one end of an intervertebral space and one embodiment of an access device configured to provide access to the intervertebral space;

FIG. 74 is a schematic lateral view of a portion of a spine with the access device of FIG. 73 applied thereto to provide access to an intervertebral space;

FIG. 75 is a schematic view similar to that of FIG. 73 illustrating one method of preparing an intervertebral space through an access device for the insertion of a replacement disc nucleus;

FIG. 76 is a schematic view similar to that of FIG. 73 illustrating one method of inserting a replacement disc nucleus into an intervertebral space through an access device

FIG. 77 is a schematic view similar to that of FIG. 73 illustrating another method of inserting a replacement disc nucleus into an intervertebral space through an access device; and

FIG. 78 is a schematic view similar to that of FIG. 73 showing additional embodiments of devices that may be used in conjunction with the insertion of a replacement disc nucleus.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF THE PREFERRED EMBODIMENTS

As should be understood in view of the following detailed description, this application is directed to apparatuses and methods for treating the spine of a patient through an access device, also referred to herein as an expandable conduit. More particularly, the systems described below provide access to surgical locations at or near the spine and provide a variety of tools useful in performing treatment of the spine. The term “surgical location” is used in its ordinary sense (i.e. a location where a surgical procedure is performed) and is a broad term and it includes locations subject to or affected by a surgery. The term “spinal location” is used in its ordinary sense (i.e. a location associated with a spine) and is a broad term and it includes locations near a spine that are sites for surgical spinal procedures. Also, the systems described herein enable a surgeon to perform a wide variety of methods as described herein.

I. Systems for Performing Procedures at a Surgical Location

Various embodiments of apparatuses and procedures described herein will be discussed in terms minimally invasive procedures and apparatuses, e.g., of endoscopic apparatuses and procedures. However, many aspects of the present invention may find use in conventional, open, and mini-open procedures. In the drawings and description which follows, the term “proximal,” as is traditional, refers to the end portion of the apparatus which is closest to the operator, while the term “distal” will refer to the end portion which is farthest from the operator.

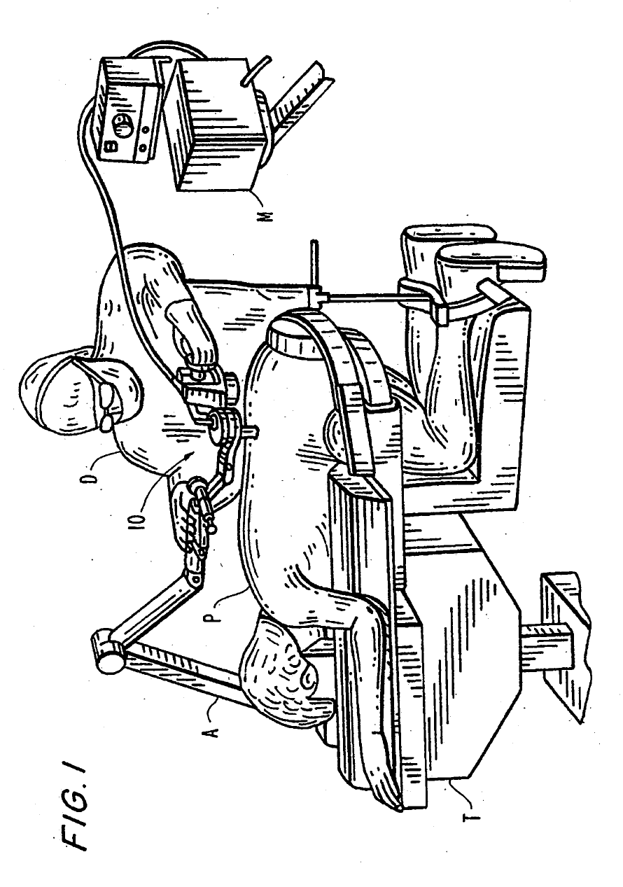

FIG. 1 shows one embodiment of a surgical system 10 that can be used to perform a variety of methods or procedures. In at least a portion of the procedure, as discussed more fully below, the patient P typically is placed in the prone position on operating table T, taking care that the abdomen is not compressed and physiological lordosis is preserved, as is known in the art. The physician D is able to access the surgical site and perform the surgical procedure with the components of the system 10, which will be described in greater detail herein. The system 10 may be supported, in part, by a mechanical support arm A, such as the type generally disclosed in U.S. Pat. No. 4,863,133, which is hereby incorporated by reference herein in its entirety. One mechanical arm of this type is manufactured by Leonard Medical, Inc., 1464 Holcomb Road, Huntington Valley, Pa., 19006.

Visualization of the surgical site may be achieved in any suitable manner, e.g., by use of a viewing element, such- as an endoscope, a camera, loupes, a microscope, direct visualization, or any other suitable viewing element, or a combination of the foregoing. In one embodiment, the viewing element provides a video signal representing images, such as images of the surgical site, to a monitor M. The viewing element may be an endoscope and camera which captures images to be displayed on the monitor M whereby the physician D is able to view the surgical site as the procedure is being performed. The endoscope and camera will be described in greater detail herein.

The systems and procedures will be described herein in connection with minimally invasive postero-lateral spinal surgery. One such method is a two level postero-lateral fixation of the spine involving the L4, L5, and S1 vertebrae. (In the drawings, the vertebrae will generally be denoted by reference letter V.) The usefulness of the apparatuses and procedures is neither restricted to the postero-lateral approach nor to the L4, L5, and S1 vertebrae, but it may be used in other anatomical approaches and other vertebra(e) within the cervical, thoracic, and lumbar regions of the spine. The procedures may be directed toward surgery involving one or more vertebral levels. It is also useful for anterior and lateral procedures. Moreover, it is believed that the invention is also particularly useful where any body structures must be accessed beneath the skin and muscle tissue of the patient, and where it desirable to provide sufficient space and visibility in order to manipulate surgical instruments and treat the underlying body structures. For example, certain features or instrumentation described herein are particularly useful for a minimally invasive procedures, e.g., arthroscopic procedures. As discussed more fully below, one embodiment of an apparatus described herein provides an expandable conduit that has an expandable distal portion. The expandable distal portion prevents or substantially prevents the expandable conduit or instruments extended therethrough to the surgical site from being dislodging or popping out of the operative site.

The system 10 includes an expandable conduit or access device that provides a internal passage for surgical instruments to be inserted through the skin and muscle tissue of the patient P to the surgical site. The expandable conduit has a wall portion defining reduced profile configuration for initial percutaneous insertion into the patient. This wall portion may have any suitable arrangement. In one embodiment, discussed in more detail below, the wall portion has a generally tubular configuration that may be passed over a dilator that has been inserted into the patient to atraumatically enlarge an opening sufficiently large to receive the expandable conduit therein.

The wall portion of the expandable conduit is subsequently expanded to an enlarged configuration, by moving against the surrounding muscle tissue to at least partially define an enlarged surgical space in which the surgical procedures will be performed. In a sense, it acts as its own dilator. The expandable conduit may also be thought of as a retractor, and may be referred to herein as such. Typically, but not by way of limitation, the distal portion expands to a greater extent than the proximal portion, because the surgical procedures are to be performed at the surgical site which is adjacent the distal portion when the expandable conduit is inserted into the patient.

While in the reduced profile configuration, the expandable conduit defines a first unexpanded configuration. Thereafter, the expandable conduit enlarges the surgical space defined thereby by engaging the tissue surrounding the conduit and displacing the tissue radially outwardly as the conduit expands. The expandable conduit may be sufficiently rigid to displace such tissue during the expansion thereof. The expandable conduit may be resiliently biased to expand from the reduced profile configuration to the enlarged configuration. In addition, the conduit may also be manually expanded by an expander device with or without one or more surgical instruments inserted therein, as will be described below. The surgical site is at least partially defined by the expanded conduit itself. During expansion, the conduit moves from the first overlapping configuration to a second overlapping configuration.

In addition to enlargement, the distal end portion of the expandable conduit may be configured for relative movement with respect to the proximal end portion in order to allow the physician to precisely position the distal end portion at the desired location. This relative movement also provides the advantage that the proximal portion of the expandable conduit nearest the physician D may remain substantially stable during such distal movement. In an exemplary embodiment, the distal portion is a separate component which is pivotably or movably attached relative to the proximal portion. In another embodiment, the distal portion is flexible or resilient in order to permit such relative movement.

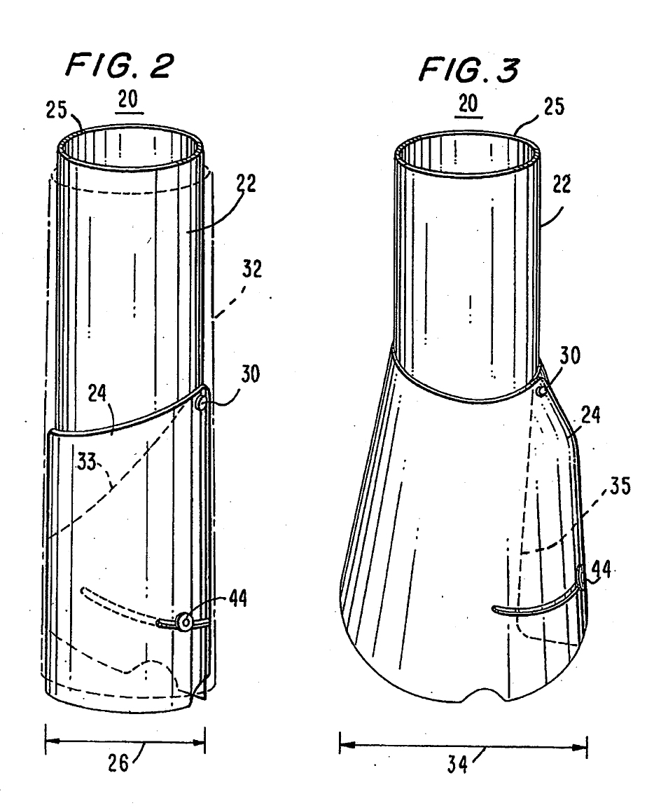

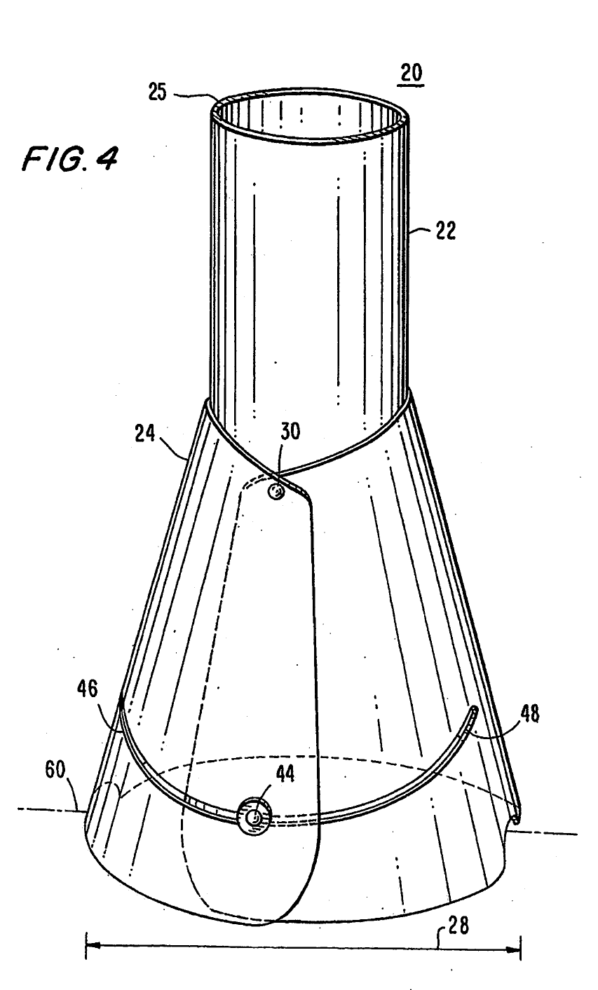

One embodiment of an expandable conduit is illustrated in FIGS. 2-6 and designated by reference number 20. The expandable conduit 20 includes a proximal wall portion. 22, which has a tubular configuration, and a distal wall portion, which is an expandable skirt portion 24. The skirt portion 24 is enlargeable from a reduced profile configuration having an initial dimension 26 and corresponding cross-sectional area (illustrated in FIG. 2), to an enlarged configuration having a dimension 28 and corresponding cross-sectional area (illustrated in FIG. 4). In one embodiment, the skirt portion 24 is attached to the proximal wall portion 22 with a rivet 30, pin, or similar connecting device to permit movement of the skirt portion 24 relative to the proximal wall portion 22.

In the illustrated embodiment, the skirt portion 24 is manufactured from a resilient material, such as stainless steel. The skirt portion 24 is manufactured so that it normally assumes an expanded configuration illustrated in FIG. 4. As illustrated in FIG. 3, the skirt portion 24 may assume an intermediate dimension 34 and corresponding cross-sectional area, which is greater than the dimension 26 of the reduced profile configuration of FIG. 2, and smaller than the dimension 28 of the enlarged configuration of FIG. 4. The skirt portion 24 may assume the intermediate configuration of FIG. 3 when deployed in the patient in response to the force of the tissue acting on the skirt portion 24. The intermediate dimension 34 will depend upon several factors, including the rigidity of the skirt portion 24, the surrounding tissue, and whether such surrounding tissue has relaxed or tightened during the course of the procedure. An outer plastic sleeve 32 (illustrated in dashed line in FIG. 2) may be provided which surrounds the expandable conduit 20 and maintains the skirt portion 24 in the reduced profile configuration. The outer sleeve 32 may have a braided polyester suture embedded within it (not shown), aligned substantially along the longitudinal axis thereof; such that when the suture is withdrawn, the outer sleeve 32 is torn, which allows the expandable conduit 20 to resiliently expand from the reduced profile configuration of FIG. 2 to the expanded configurations of FIGS. 3-4. While in the reduced profile configuration of FIG. 2, the skirt portion 24 defines a first overlapping configuration 33, as illustrated by the dashed line. As the skirt portion 24 resiliently expands, the skirt portion 24 assumes the expanded configuration, as illustrated in FIGS. 3-4.

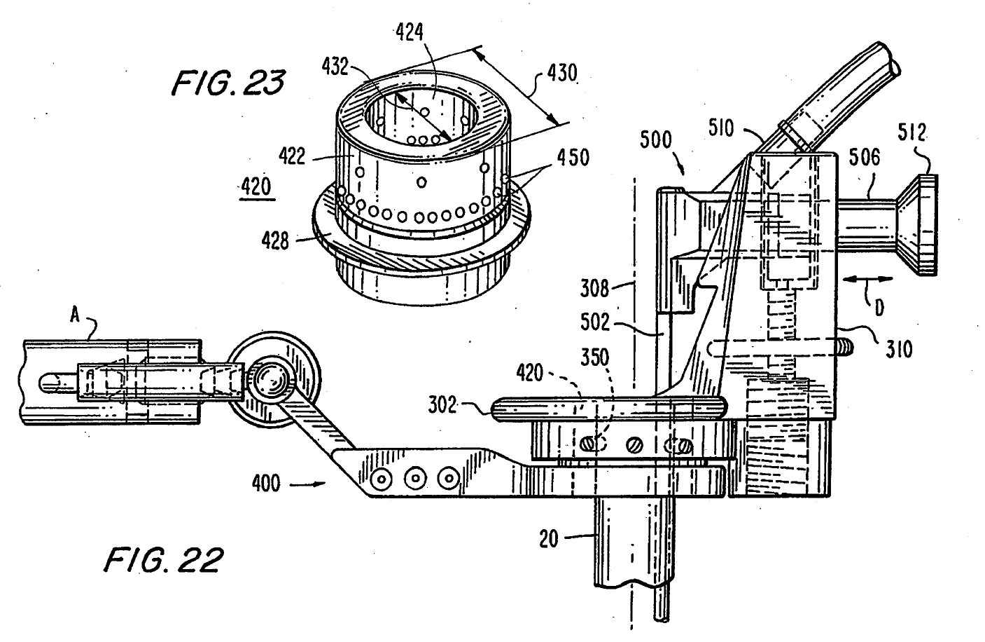

The skirt portion 24 is sufficiently rigid that it is capable of displacing the tissue surrounding the skirt portion 24 as it expands. Depending upon the resistance exerted by surrounding tissue, the skirt portion is sufficiently rigid to provide some resistance against the tissue to remain in the configurations of FIGS. 3-4. Moreover, the expanded configuration of the skirt portion 24 is at least partially supported by the body tissue of the patient. The rigidity of the skirt portion 24 and the greater expansion at the distal portion. creates a stable configuration that is at least temporarily stationary in the patient, which frees the physician from the need to actively support the conduit 20 until an endoscope mount platform 300 and a support arm 400 are subsequently added in one embodiment (see FIGS. 21-22).

The skirt portion 24 of the expandable conduit 20 is illustrated in an initial flattened configuration in FIG. 5. The skirt portion 24 may be manufactured from a sheet of stainless steel having a thickness of about 0.007 inches. In various embodiments, the dimension 28 of the skirt portion 24 is about equal to or greater than 50 mm, is about equal to or greater than 60 mm, is about equal to or greater than 70 mm, is about equal to or greater than 80 mm, or is any other suitable size, when the skirt portion 24 is in the enlarged configuration. In one embodiment, the dimension 28 is about 63 mm, when the skirt portion 24 is in the enlarged configuration. As discussed above, the unrestricted shape of the skirt portion 24 preferably is a circular or an oblong shape. The skirt portion 24 may also take on an oval shape, wherein the dimension 28 would define a longer dimension the skirt portion 24 and would be about 85 mm in one embodiment. In another embodiment, the skirt portion 24 has an oval shape and the dimension 28 defines a longer dimension of the skirt portion 24 and would be about 63 mm. An increased thickness, e.g., about 0.010 inches, may be used in connection with skirt portions having a larger diameter, such as about 65 mm. Other materials, such as nitinol or plastics having similar properties, may also be useful.

As discussed above, the skirt portion 24 is attached to the proximal wall portion 22 with a pivotable connection, such as rivet 30. A pair of rivet holes 36 are provided in the skirt portion 24 to receive the rivet 30. The skirt portion 24 also has two free ends 38 and 40 in one embodiment that are secured by a slidable connection, such as second rivet 44 (not shown in FIG. 5, illustrated in FIGS. 2-4). A pair of complementary slots 46 and 48 are defined in the skirt portion 24 adjacent the free ends 38 and 40. The rivet 44 is permitted to move freely within the slots 46 and 48. This slot and rivet configuration allows the skirt portion 24 to move between the reduced profile configuration of FIG. 2 and the enlarged or expanded configurations of FIGS. 3-4. The use of a pair of slots 46 and 48 reduces the risk of the “button-holing” of the rivet 44, e.g., a situation in which the opening of the slot becomes distorted and-enlarged such that the rivet may slide out of the slot, and cause failure of the device. However, the likelihood of such occurrence is reduced in skirt portion 24 because each of the slots 46 and 48 in the double slot configuration has a relatively shorter length than a single slot configuration. Being shorter, the slots 46, 48 are less likely to be distorted to the extent that a rivet may slide out of position. In addition, the configuration of rivet 44 and slots 46 and 48 permits a smoother operation of enlarging and reducing the skirt portion 24, and allows the skirt portion 24 to expand to span as many as three vertebrae, e.g., L4, L5, and S1, to perform multi-level fixation alone or in combination with a variety of other procedures, as discussed below.

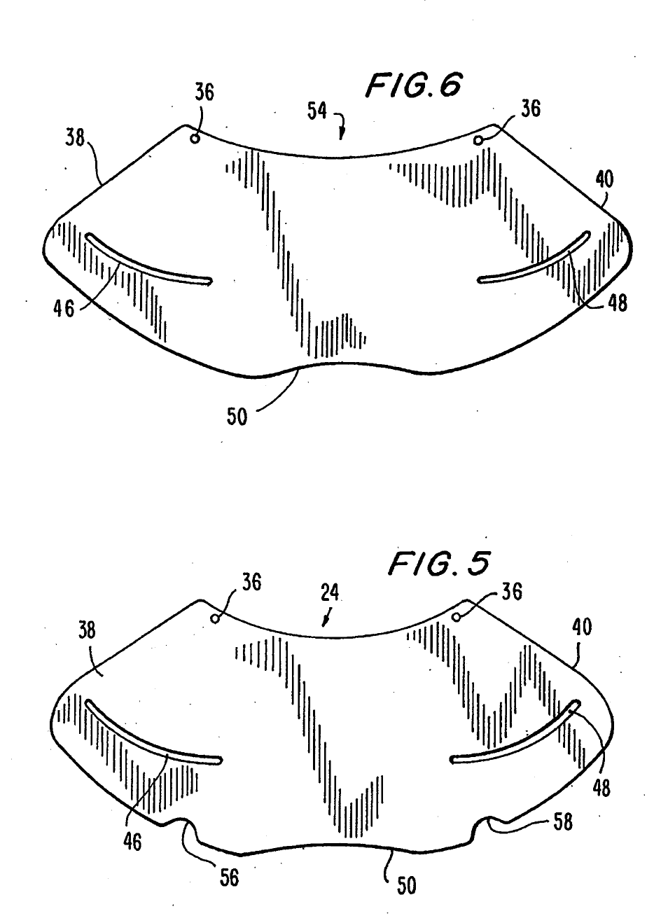

An additional feature of the skirt portion 24 is the provision of a shallow concave profile 50 defined along the distal edge of the skirt portion 24, which allows for improved placement of the skirt portion 24 with respect to the body structures and the surgical instruments defined herein. In one embodiment, a pair of small scalloped or notched portions 56 and 58, are provided, as illustrated in FIG. 5. When the skirt portion 24 is assembled, the notched portions 56 and 58 are oriented in the cephcaudal direction (indicated by an arrow 60 in FIG. 4) and permit instrumentation, such as an elongated member 650 used in a fixation procedure. (described in detail below), to extend beyond the area enclosed by the skirt portion 24 without moving or raising the skirt portion 24 from its location to allow the elongated member 650 to pass under the skirt portion 24. The notched portions 56, 58 are optional, as illustrated in connection with another embodiment of an expandable conduit 54, illustrated in FIG. 6, and may be eliminated where the physician deems the notches to be unnecessary for the procedures to be performed (e.g., where fixation does not require extended access, as discussed more fully below.)

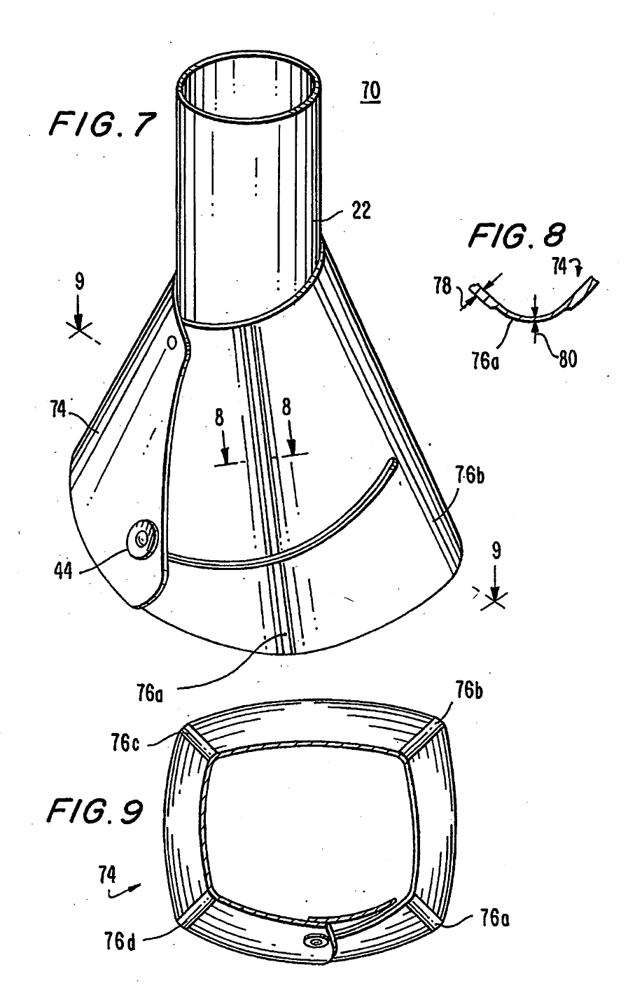

As illustrated in FIG. 4, the skirt portion 24 may be expanded to a substantially conical configuration having a substantially circular or elliptical profile. In another embodiment, features may be provided on the skirt portion which facilitate the bending of the skirt portion at several locations to provide a pre-formed enlarged configuration. For example, another embodiment of an expandable conduit 70, illustrated in FIGS. 7-9, provides a skirt portion 74 that has four sections 76a, 76b, 76c, 76d having a reduced thickness. For a skirt portion 74 having a thickness 78 of about 0.007 inches, reduced thickness sections 76a, 76b, 76c, 76d may have a thickness 80 of about 0.002-0.004 inches (FIG. 8). The reduced thickness sections 76a, 76b, 76c, 76d may have a width 82 of about 1-5 mm. The thickness 78 of the skirt portion 74 may be reduced by milling or grinding, as is known in the art. When the skirt portion 74 is opened, it moves toward a substantially rectangular configuration, as shown in FIG. 9, subject to the resisting forces of the body tissue. In another embodiment (not shown), a skirt portion may be provided with two reduced thickness sections (rather than the four reduced thickness sections of skirt 74) which would produce a substantially “football”-shaped access area.

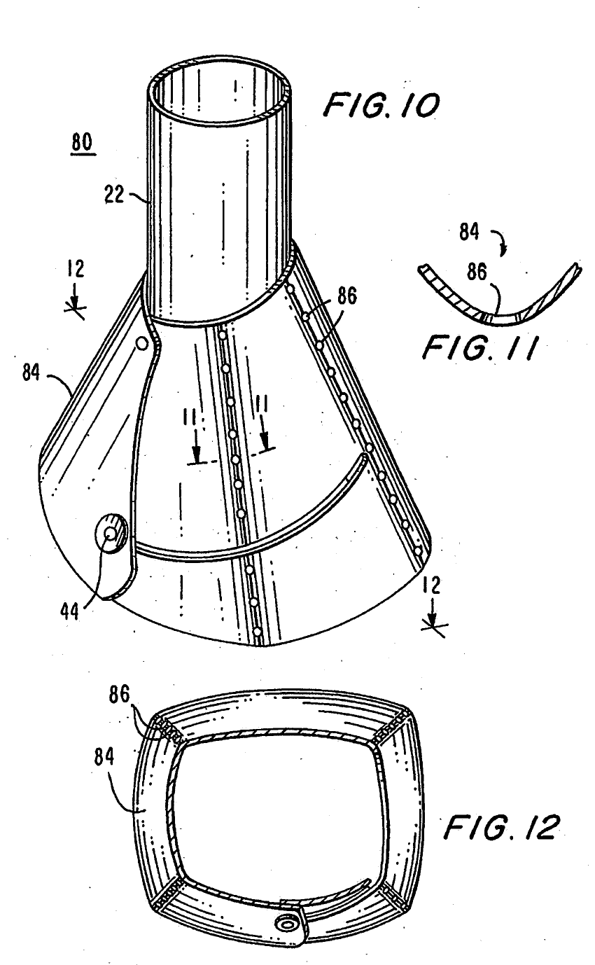

FIGS. 10-12 show another embodiment of an expandable conduit 80. The expandable conduit 80 has a skirt portion 84 with a plurality of perforations 86. The perforations 86 advantageously increase the flexibility at selected locations. The size and number of perforations 86 may vary depending upon the desired flexibility and durability. In another embodiment, the skirt portion 84 may be scored or otherwise provided with a groove or rib in order to facilitate the bending of the skirt portion at the desired location.

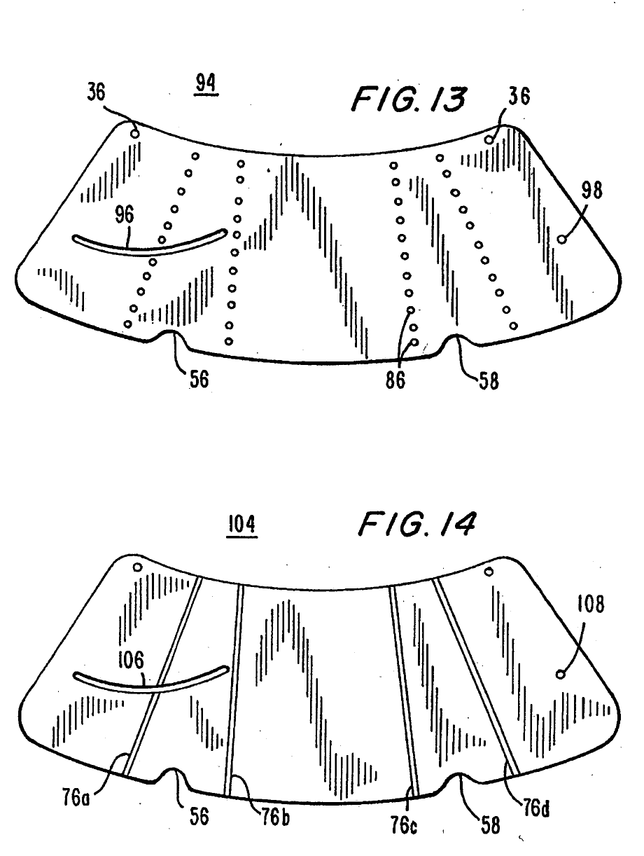

FIG. 13 illustrates another embodiment of an expandable conduit that has a skirt portion 94 having one slot 96 and an aperture 98. A rivet (not shown) is stationary with respect to the aperture 98 and slides within the slot 96. FIG. 14 illustrates another embodiment of an expandable conduit that has a skirt portion 104 that includes an aperture 108. The apertures 108 receives a rivet (not shown) that slides within elongated slot 106.

Further details of the expandable conduit are described in U.S. Pat. No. 6,187,00, and in U.S. patent application Ser. No. 09/772,605, filed Jan. 30, 2001, U.S. application Ser. No. 10/361,887 filed Feb. 10, 2003, and application Ser. No. 10/280,489 filed Oct. 25, 2002, which are hereby incorporated herein by reference in their entirety.

In one embodiment of a procedure, an early stage involves determining a point in the skin of the patient at which to insert the expandable conduit. The access point preferably corresponds to the posterior-lateral aspects of the spine. Manual palpation and Anterior-Posterior (AP) fluoroscopy may be used to determine preferred or optimal locations for forming an incision in the skin of the patient. In one embodiment, the expandable conduit 20 preferably is placed midway (in the cephcaudal direction) between the L4 through S1 vertebrae, centrally about 4-7 cm from the midline of the spine.

After the above-described location is determined, an incision is made at the location. A guide wire (not shown) is introduced under fluoroscopic guidance through the skin, fascia, and muscle to the approximate surgical site. A series of dilators is used to sequentially expand the incision to the desired width, about 23 mm in one procedure, without damaging the structure of surrounding tissue arid muscles. A first dilator is placed over the guide wire, which expands the opening. The guide wire is then subsequently removed. A second dilator that is slightly larger than the first dilator is placed over the first dilator, which expands the opening further. Once the second dilator is in place, the first dilator is subsequently removed. This process of (1) introducing a next-larger-sized dilator coaxially over the previous dilator and (2) subsequently removing the previous dilator when the next-larger-sized dilator is in place continues until an opening of the desired size is created in the skin, muscle, and subcutaneous tissue. In one embodiment of the method, desired opening size is about 23 mm. (Other dimensions of the opening, e.g., about 20 mm, 27 mm, 30 mm, etc., are also useful with this apparatus in connection with spinal surgery, and still other dimensions are contemplated.)

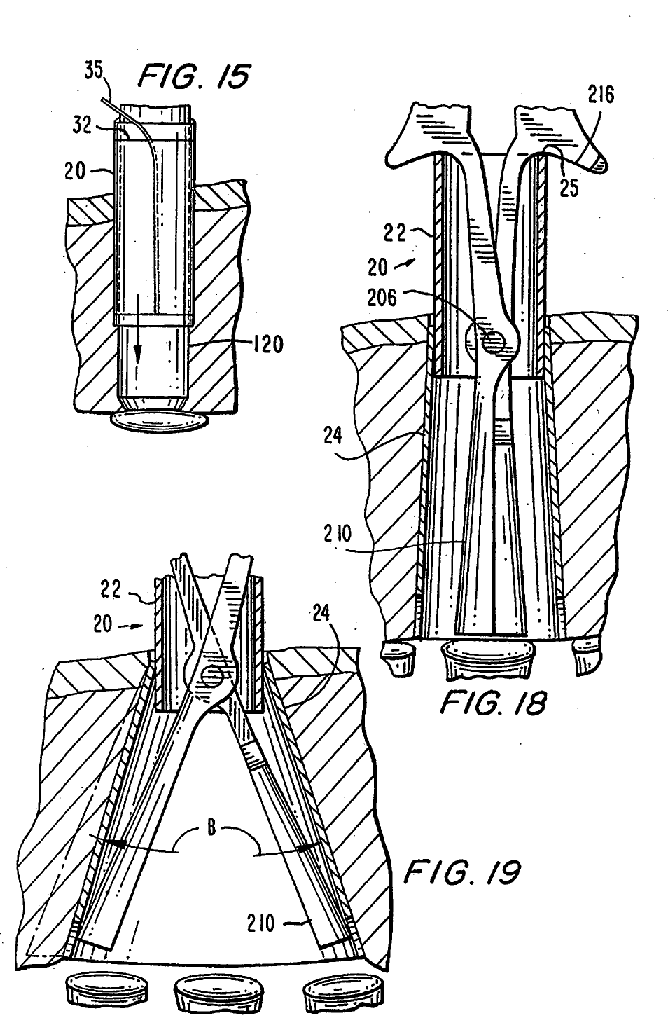

FIG. 15 shows that following placement of a dilator 120, which is the largest dilator in the above-described dilation process, the expandable conduit 20 is introduced in its reduced profile configuration and positioned in a surrounding relationship over the dilator 120. The dilator 120 is subsequently removed from the patient, and the expandable conduit 20 is allowed to remain in position.

Once positioned in the patient, the expandable conduit 20 may be enlarged to provide a passage for the insertion of various surgical instruments and to provide an enlarged space for performing the procedures described herein. As described above, the expandable conduit may achieve the enlargement in several ways. In one embodiment, a distal portion of the conduit may be enlarged, and a proximal portion may maintain a constant diameter. The relative lengths of the proximal portion 22 and the skirt portion 24 may be adjusted to vary the overall expansion of the conduit 20. Alternatively, such expansion may extend along the entire length of the expandable conduit 20. In one embodiment of a procedure, the expandable conduit 20 may be expanded by removing a suture 35 and tearing the outer sleeve 32 surrounding the expandable conduit 20, and subsequently allowing the skirt portion 24 to resiliently expand towards its fully expanded configuration as (illustrated in FIG. 4) to create an enlarged surgical space from the L4 to the S1 vertebrae. The resisting force exerted on the skirt portion 24 may result in the skirt portion 24 assuming the intermediate configuration illustrated in FIG. 3. Under many circumstances, the space created by the skirt portion 24 in the intermediate configuration is a sufficiently large working space to perform the procedure described herein. Once the skirt portion 24 has expanded, the rigidity and resilient characteristics of the skirt portion 24 allow the expandable conduit 20 to resist closing to the reduced profile configuration of FIG. 2 and to at least temporarily resist being expelled from the incision. These characteristics create a stable configuration for the conduit 20 to remain in position in the body, supported by the surrounding tissue. It is understood that additional support may be needed, especially if an endoscope is added.

According to one embodiment of a procedures, the expandable conduit 20 may be further enlarged at the skirt portion 24 using an expander apparatus to create a surgical access space. An expander apparatus useful for enlarging the expandable conduit has a reduced profile configuration and an enlarged configuration. The expander apparatus is inserted into the expandable conduit in the reduced profile configuration, and subsequently expanded to the enlarged configuration. The expansion of the expander apparatus also causes the expandable conduit to be expanded to the enlarged configuration. In some embodiments, the expander apparatus may increase the diameter of the expandable conduit along substantially its entire length in a conical configuration. In other embodiments, the expander apparatus expands only a distal portion of the expandable conduit, allowing a proximal portion to maintain a constant diameter.

In addition to expanding the expandable conduit, the expander apparatus may also be used to position the distal portion of the expandable conduit at the desired location for the surgical procedure. The expander engages an interior wall of the expandable conduit, and moves the conduit to the proper location. For the embodiments in which the distal portion of the expandable conduit is relatively movable with respect to the proximal portion, the expander apparatus is useful to position the distal portion without substantially disturbing the proximal portion.

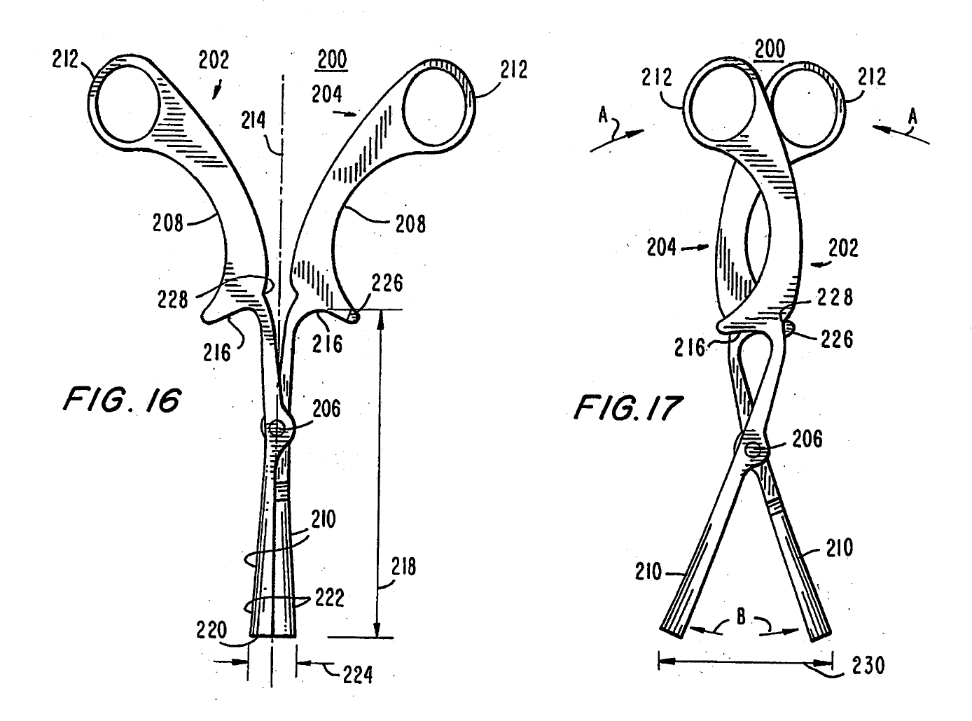

In some procedures, an expander apparatus is used to further expand the skirt portion 24 towards the enlarged configuration (illustrated in FIG. 4). The expander apparatus is inserted into the expandable conduit, and typically has two or more members which are movable to engage the interior wall of the skirt portion 24 and apply a force sufficient to further expand the skirt portion 24. FIGS. 16 and 17 show one embodiment of an expander apparatus 200 that has a first component 202 and a second component 204 a first component 202 and a second component 204 of the expander apparatus 200 are arranged in a tongs-like configuration and are pivotable about a pin 206. The first and second components 202 and 204 are typically constructed of steel having a thickness of about 9.7 mm. Each of the first and second components 202 and 204 has a proximal handle portion 208 and a distal expander portion 210. Each proximal handle portion 208 has a finger grip 212 that may extend transversely from an axis, e.g., a longitudinal axis 214, of the apparatus 200. The proximal handle portion 208 may further include a stop element, such as flange 216, that extends transversely from the longitudinal axis 214. The flange 216 is dimensioned to engage the proximal end 25 of the expandable conduit 20 when the apparatus 200 is inserted a predetermined depth. This arrangement provides a visual and tactile indication of the proper depth for inserting the expander apparatus 200. In one embodiment, a dimension 218 from the flange 216 to the distal tip 220 is about 106 mm. The dimension 218 is determined by the typical depth of the body structures beneath the skin surface at which the surgical procedure is being performed. The distal portions 210 are each provided with an outer surface 222 for engaging the inside wall of the skirt portion 24. The outer surface 222 is a frusto-conical surface in one embodiment. The expander apparatus 200 has an unexpanded distal width 224 at the distal tip 220 that is about 18.5 mm in one embodiment.

In use, the finger grips 212 are approximated towards one another, as indicated by an arrow A in FIG. 17, which causes the distal portions 210 to move to the enlarged configuration, as indicated by arrows B. The components 202 and 204 are also provided with a cooperating tab 226 and shoulder portion 228 which are configured for mutual engagement when the distal portions 210 are in the expanded configuration. In the illustrated embodiment, the expander apparatus 200 has an expanded distal width 230 that extends between the distal portions 210. The expanded distal width 230 can be about 65 mm or less, about as large as 83 mm or less, or any other suitable width. The tab 226 and shoulder portion 228 together limit the expansion of the expander apparatus 200 to prevent expansion of the skirt portion 24 of the expandable conduit 20 beyond its designed dimension, and to minimize trauma to the underlying tissue. Further details of the expander apparatus are described in U.S. patent application Ser. No. 09/906,463. filed Jul. 16,.2001, which is hereby incorporated by reference herein in their entirety.

When the expandable conduit 20 is inserted into the patient and the outer sleeve 32 is removed, the skirt portion 24 expands to a point where the outward resilient expansion of the skirt portion 24 is balanced by the force of the surrounding tissue. The surgical space defined by the conduit may be sufficient to perform any of a number of surgical procedures or combination of surgical procedures described herein. However, if it is desired to expand the expandable conduit 20 further, the expander apparatus 200 may be inserted into the expandable conduit 20 in the reduced profile configuration until the shoulder portions 216 are in approximation with the proximal end 25 of the skirt portion 24 of the expandable conduit 20, as shown in FIG. 18.

FIG. 18 shows the expander apparatus 200 is inserted in the expandable conduit 20 in the reduced profiled configuration. Expansion of the expander apparatus 200 is achieved by approximating the handle portions 212 (not shown in FIG. 18), which causes the distal portions 210 of the expander apparatus 200 to move to a spaced apart configuration. As the distal portions 210 move apart and contact the inner wall of the skirt portion 24, the skirt portion 24 is expanded by allowing the rivet 44 to slide within the slots 46 and 48 of the skirt portion 24. When the distal portions 210 reach the maximum expansion of the skirt portion 24 (illustrated by a dashed line in FIG. 19), the tab 226 and shoulder portion 228 of the expander apparatus 200 come into engagement to prevent further expansion of the tong portions (as illustrated in FIG. 17). The conduit 20 may be alternatively further expanded with a balloon or similar device.

A subsequent, optional step in the procedure is to adjust the location of the distal portion of the expandable conduit 20 relative to the body structures to be operated on. For example, the expander apparatus 200 may also be used to engage the inner wall of the skirt portion 24 of the expandable conduit 20 in order to move the skirt portion 24 of the expandable conduit 20 to the desired location. For an embodiment in which the skirt portion 24 of the expandable conduit 20 is relatively movable relative to the proximal portion, e.g. by use of the rivet 30, the expander apparatus 200 is useful to position the skirt portion 24 without substantially disturbing the proximal portion 22 or the tissues closer to the skin surface of the patient. As will be described below, the ability to move the distal end portion, e.g., the skirt portion 24, without disturbing the proximal portion is especially beneficial when an additional apparatus is mounted relative to the proximal portion of the expandable conduit, as described below.

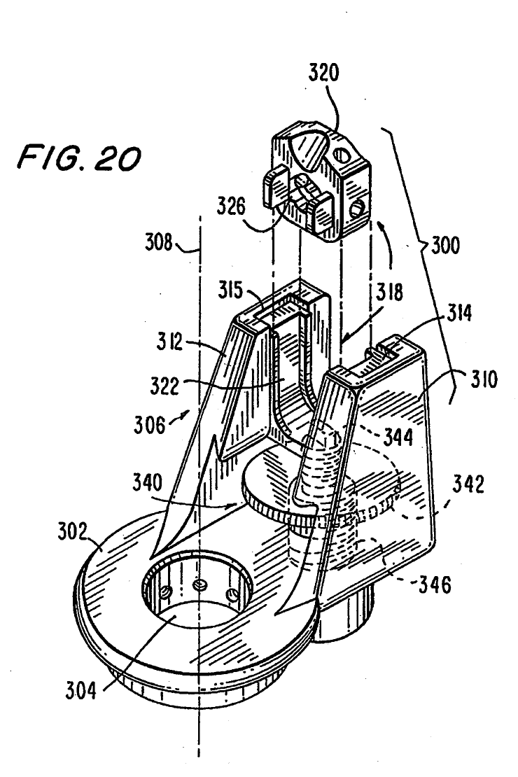

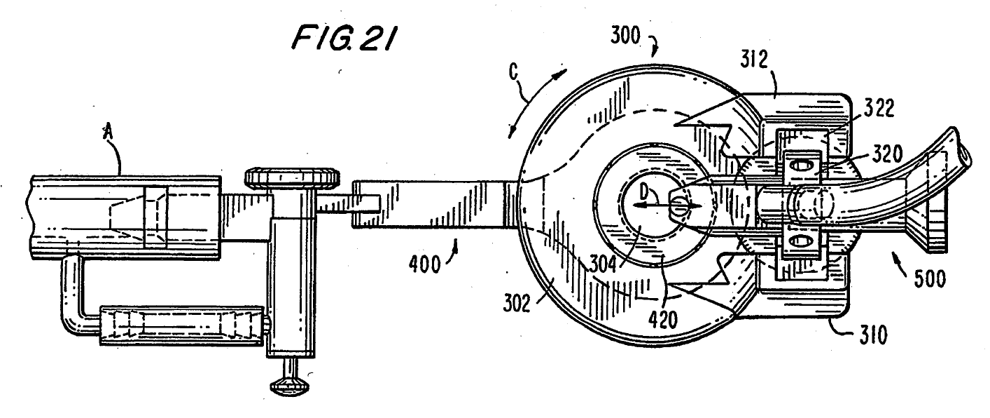

An endoscope mount platform 300 and indexing arm 400 provide securement of an endoscope 500 on the proximal end 25 of the expandable conduit 20 for remotely viewing the surgical procedure, as illustrated in FIGS. 20-23. The endoscope mount platform 300 may also provide several other functions during the surgical procedure. The endoscope mount platform 300 includes a base 302 that extends laterally from a central opening 304 in a general ring-shaped configuration. The base 302 provides an aid for the physician, who is primarily viewing the procedure by observing a monitor, when inserting surgical instruments into the central opening 304. For example, the size of the base 302 provides visual assistance (as it may be observable in the physician's peripheral vision) as well as provides tactile feedback as the instruments are lowered towards the central opening 304 and into the expandable conduit 20.

The endoscope mount platform 300 further provides a guide portion 306 that extends substantially parallel to a longitudinal axis 308 away from the central opening 304. The base 302 is typically molded as one piece with the guide portion 306. The base 302 and guide portion 306 may be constructed as a suitable polymer such as polyetheretherketone (PEEK).

The guide portion 306 includes a first upright member 310 that extends upward from the base 302 and a second upright member 312 that extends upward from the base 302. The upright members 310, 312 each have a respective vertical grooves 314 and 315 that can slidably receive an endoscopic mount assembly 318.

The endoscope 500 (not shown in FIG. 20) is movably mounted to the endoscope mount platform 300 by the endoscope mount assembly 318. The endoscope mount assembly 318 includes an endoscope mount 320 and a saddle unit 322. The saddle unit 322 is slidably mounted is within the grooves 314 and 315 in the upright members 310 and 312. The endoscope mount 320 receives the endoscope 500 through a bore 326 which passes through the endoscope mount 320. Part of the endoscope 500 may extend through the expandable conduit 20 substantially parallel to longitudinal axis 308 into the patient's body 130.

The endoscope mount 320 is removably positioned in a recess 328 defined in the substantially “U”-shaped saddle unit 322, which is selectively movable in a direction parallel to the longitudinal axis 308 in order to position the endoscope 500 at the desired height within the expandable conduit 20 to provide a zoom feature to physician's view of the surgical procedure.

A screw mechanism 340 is positioned on the base 302 between the upright members 310 and 312, and is used to selectively move the saddle unit 322, and the endoscope mount 320 and the endoscope 500 which are supported by the saddle unit 322. The screw mechanism 340 comprises a thumb wheel 342 and a spindle 344. The thumb wheel 343 is rotatably mounted in a bore in the base 302. The thumb wheel 342 has an external thread 346 received in a cooperating thread in the base 302. The spindle 344 is mounted for movement substantially parallel to the central axis 308. The spindle 344 has a first end received in a rectangular opening in the saddle unit 322, which inhibits rotational movement of the spindle 344. The second end of the spindle 344 has an external thread which cooperates with an internal thread formed in a bore within the thumb wheel 342. Rotation of the thumb wheel 342 relative to the spindle 344, causes relative axial movement of the spindle unit 344 along with the saddle unit 322. Further details of the endoscope mount platform are described in U.S. patent application Ser. No. 09/491,808 filed Jan. 28, 2000, application Ser. No. 09/821,297 filed Mar. 29, 2001, and application Ser. No. 09/940,402 filed Aug. 27, 2001.

FIG. 21-23 show that the endoscope mount platform 300 is mountable to the support arm 400 in one embodiment. The support arm 400, in turn, preferably is mountable to mechanical support, such as mechanical support arm A, discussed above in connection with FIG. 1. The support arm 400 rests on the proximal end 25 of the expandable conduit 20. The support arm 400 includes an indexing collar 420, which is received in the central opening 304 of the base 302 of endoscope mount platform 300. The indexing collar 420 is substantially toroidal in section and has an outer peripheral wall surface 422, an inner wall surface 424, and a wall thickness 426 that is the distance between the wall surfaces 422, 424. The indexing collar 420 further includes a flange 428, which supports the indexing collar 420 on the support arm 400.

The collars 420 advantageously make the surgical system 10 a modular in that different expandable conduits 20 may be used with a single endoscope mount platform 300. For example, expandable conduits 20 of different dimensions may be supported by providing of indexing collars 420 to accommodate each conduit size while using a single endoscope mount platform 300. The central opening 304 of the endoscope mount platform 300 has constant dimension, e.g., a diameter of about 32.6 mm. An appropriate indexing collar 420 is selected, e.g., one that is appropriately sized to support a selected expandable conduit 20. Thus the outer wall 422 and the outer diameter 430 are unchanged between different indexing collars 420, although the inner wall 424 and the inner diameter 432 vary to accommodate differently sized conduits 20.

The indexing collar 420 is mounted to the proximal portion of the expandable conduit 20 and allows angular movement of the endoscope mount platform 300 with respect thereto about the longitudinal axis 308 (as indicated by an arrow C in FIG. 21). The outer wall 422 of the index collar 420 includes a plurality of hemispherical recesses 450 that can receive one or more ball plungers 350 on the endoscope mount platform 300 (indicated in dashed line.) This arrangement permits the endoscope mount platform 300, along with the endoscope 500, to be fixed in a plurality of discrete angular positions. Further details of the support arm and indexing collar are described in U.S. Pat. No. 6,361,488, issued Mar. 26, 2002, U.S. Pat. No. 6,530,880 issued Mar. 11, 2003, and application Ser. No. 09/940,402 filed Aug. 27, 2001.

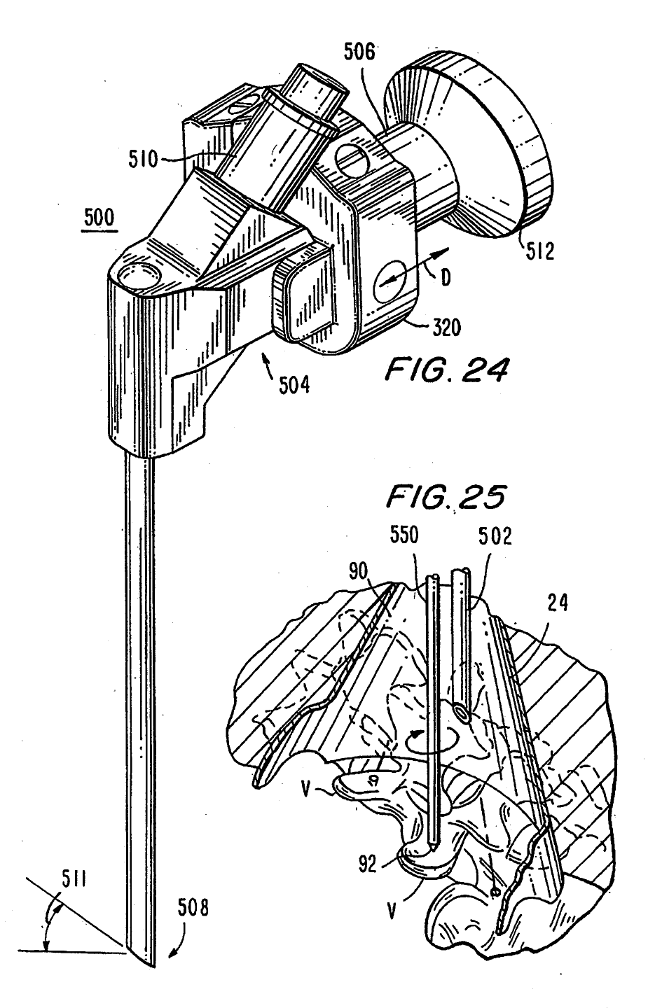

FIG. 24 shows one embodiment of the endoscope 500, which has an elongated configuration that extends into the expandable conduit 20 in order to view the surgical site. In particular, the endoscope 500 has an elongated rod portion 502 and a body portion 504 which is substantially perpendicular thereto. In the illustrated embodiment, the rod portion 502 of endoscope 500 has a diameter of about 4 mm and a length of about 106 mm. Body portion 504 may define a tubular portion 506 which is configured to be slidably received in the bore 326 of endoscope mount 320 as indicated by an arrow D. The slidable mounting of the endoscope 500 on the endoscope mount platform 300 permits the endoscope 500 to adjust to configurations that incorporate different conduit diameters. Additional mobility of the endoscope 500 in viewing the surgical site may be provided by rotating the endoscope mount platform 300 about the central axis 308 (as indicated by arrow C in FIG. 21).

The rod portion 502 supports an optical portion (not shown) at a distal end 508 thereof, which may define a field of view of about 105 degrees and a direction of view 511 of about 25-30 degrees. An eyepiece 512 is positioned at an end portion of the body portion 504. A camera (not shown) preferably is attached to the endoscope 500 adjacent the eyepiece 512 with a standard coupler unit. A light post 510 supplies illumination to the surgical site at the distal end portion 508. A preferred camera for use in the system and procedures described herein is a three chip unit that provides greater resolution to the viewed image than a single chip device.

A subsequent stage in the procedure involves placing the support arm 400 and the endoscope mount platform 300 on the proximal portion, e.g., the proximal end 25, of the expandable conduit 20 (FIGS. 1 and 22), and mounting of the endoscope 500 on the endoscope mount platform 300. A next step is insertion of one or more surgical instruments into the expandable conduit 20 to perform the surgical procedure on the body structures at least partially within the operative space defined by the expanded portion of the expandable conduit. FIG. 25 shows that in one method, the skirt portion 24 of expandable conduit 20 at least partially defines a surgical site or operative space 90 in which the surgical procedures described herein may be performed. Depending upon the overlap of the skirt portion, the skirt portion may define a surface which is continuous about the circumference or which is discontinuous having one or more gaps where the material of the skirt portion does not overlap.

One procedure performable through the expandable conduit 20, described in greater detail below, is a two-level spinal fixation. Surgical instruments inserted into the expandable conduit may be used for debridement and decortication. In particular, the soft tissue, such as fat and muscle, covering the vertebrae may be removed in order to allow the physician to visually identify the various “landmarks,” or vertebral structures, which enable the physician to locate the location for attaching a fastener, such a fastener 600, discussed below, or other procedures, as will be described herein. Allowing visual identification of the vertebral structures enables the physician to perform the procedure while viewing the surgical area through the endoscope, microscope, loupes, etc., or in a conventional, open manner.

Tissue debridement and decortication of bone are completed using one or more debrider blades, bipolar sheath, high speed burr, and additional conventional manual instruments. The debrider blades are used to excise, remove and aspirate the soft tissue. The bipolar sheath is used to achieve hemostasis through spot and bulk tissue coagulation. The debrider blades and bipolar sheath are described in greater detail in U.S. Pat. No. 6,193,715, assigned to Medical Scientific, Inc., which is hereby incorporated by reference in its entirety herein. The high speed burr and conventional manual instruments are also used to continue to expose the structure of the vertebrae.

A subsequent stage is the attachment of fasteners to the vertebrae V. Prior to attachment of the fasteners, the location of the fastener attachment is confirmed. In the exemplary embodiment, the pedicle entry point of the L5 vertebrae is located using visual landmarks as well as lateral and A/P fluoroscopy, as is known in the art. With continued reference to FIG. 25, the entry point 92 is prepared with an awl 550. The pedicle hole 92 is completed using instruments known in the art such as a straight bone probe, a tap, and a sounder. The sounder, as is known in the art, determines whether the hole that is made is surrounded by bone on all sides, and that there has been no perforation of the pedicle wall.

After hole in the pedicle is provided at the entry point 92 (or at any point during the procedure), an optional step is to adjust the location of the distal portion of the expandable conduit 20. This may be performed by inserting the expander apparatus 200 into the expandable conduit 20, expanding the distal portions 210, and contacting the inner wall of the skirt portion 24 to move the skirt portion 24, to the desired location. This step may be performed while the endoscope 500 is, positioned within the expandable conduit 20, and without substantially disturbing the location of the proximal portion of the expandable conduit 20 to which the endoscope mount platform 300 may be attached.

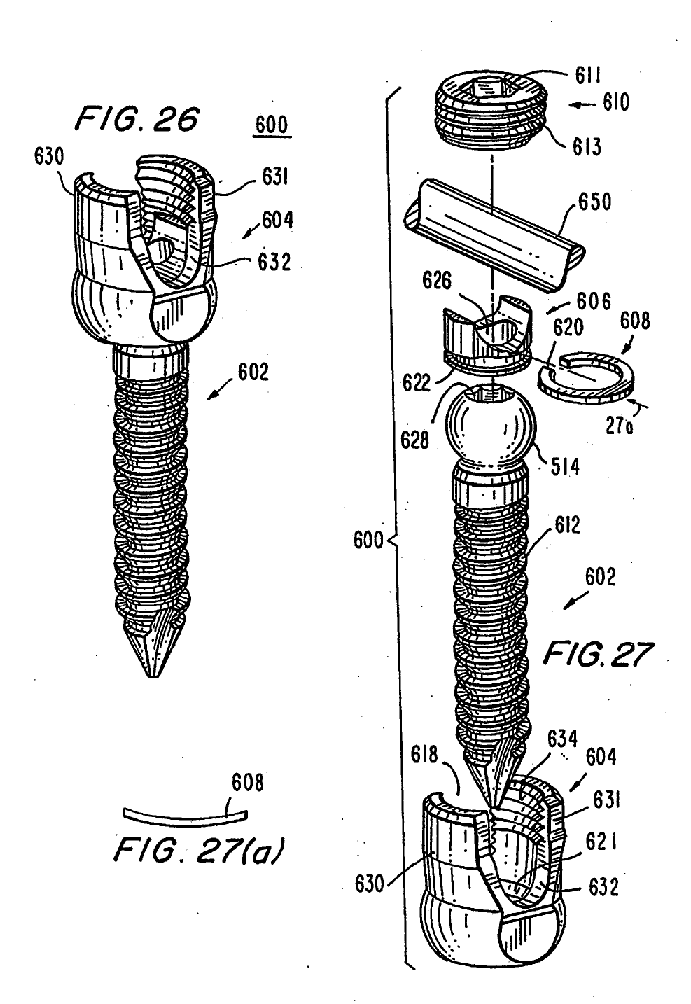

FIGS. 26-27 illustrate a fastener 600 that is particularly applicable in a procedures involving fixation. The fastener 600 is described in greater detail in U.S. patent application Ser. No. 10/075,668, filed Feb. 13, 2002 and application Ser. No. 10/087,489, filed Mar. 1, 2002, which are hereby incorporated by reference in their entirety. Fastener 600 includes a screw portion 602, a housing 604, a spacer member 606, a biasing member 608, and a clamping member, such as a cap screw 610. The screw portion 602 has a distal threaded portion 612 and a proximal, substantially spherical joint portion 614. The threaded portion 612 is inserted into the hole 92 in the vertebrae, as will be described below. The substantially spherical joint portion 614 is received in a substantially annular, part spherical recess 616 in the housing 604 in a ball and socket joint relationship (see also FIG. 29).

As illustrated in FIG. 27, the fastener 600 is assembled by inserting the screw portion 602 into a bore in a passage 618 in the housing 604, until the joint portion 614 engages the annular recess 616. The screw portion 602 is retained in the housing 604 by the spacer member 606 and biasing member 608. The biasing member 608 provides a biasing force to drive the spacer member 606 in frictional engagement with the joint portion 614 of the screw member 602 and the annular recess 616 of the housing 604. The biasing provided by the biasing member 602 frictionally maintains the relative positions of the housing 604 with respect to the screw portion 602. The biasing member 608 is selected such that biasing force prevents unrestricted movement of the housing 604 relative to the screw portion 602. However, the biasing force is insufficient to resist the application of force by a physician to move the housing 604 relative to the screw portion 602. In other words, this biasing force is strong enough maintain the housing 604 stationary relative to the screw portion 602, but this force may be overcome by the physician to reorient the housing 604 with respect to the screw member 602, as will be described below.

In the illustrated embodiment, the biasing member 608 is a resilient ring having a gap 620, which permits the biasing member 608 to radially contract and expand. FIG. 27(a) illustrates that the biasing member 608 may have an arched shape, when viewed end-on. The arched shape of the spring member 608 provides the biasing force, as will be described below. The spacer member 606 and the biasing member 608 are inserted into the housing 604 by radially compressing the biasing member into an annular groove 622 in the spacer member 606. The spacer member 606 and the biasing member 608 are slid into the passage 618 until the distal surface of the spacer member 606 engages the joint portion 614 of the screw portion 602, and the biasing member 608 expands radially into the annular groove 622 in the housing 604. The annular groove 622 in the housing 604 has a dimension 623 which is smaller than the uncompressed height of the arched shape of the biasing member 608. When the biasing member 608 is inserted in the annular groove 620, the biasing member 608 is flattened against its normal bias, thereby exerting the biasing force to the spacer member 606. It is understood that similar biasing members, such as coiled springs, belleville washers, or the like may be used to supply the biasing force described herein.

The spacer member 606 is provided with a longitudinal bore 626, which provides access to a hexagonal recess 628 in the proximal end of the joint portion 614 of the screw member 602. The proximal portion of the housing 604 includes a pair of upright members 630 and 631 that are separated by substantially “U”-shaped grooves 632. A recess for receiving elongated member 650 is defined by the pair of grooves 632 between upright member 630 and 631. Elongated member 650 to be placed distally into the housing 604 in an orientation substantially transverse to the longitudinal axis of the housing 604, as will be described below. The inner walls of he upright members 630 and 631 are provided with threads 634 for attachment of the cap screw 610 by threads 613 therein.

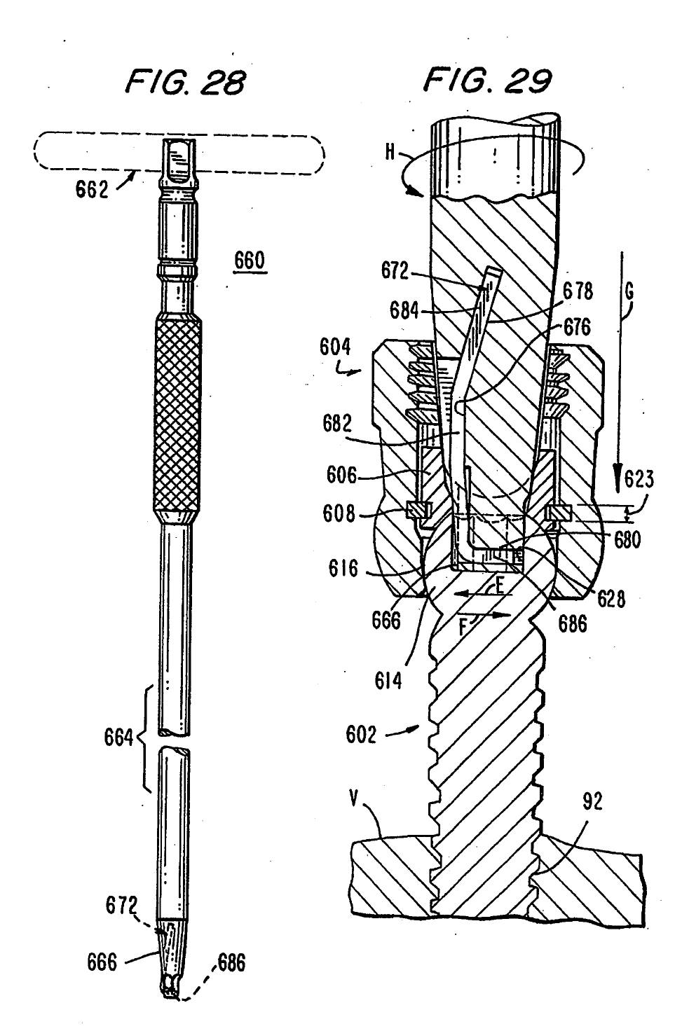

The fastener 600 is inserted into the expandable conduit 20 and guided to the prepared hole 92 in the vertebrae as a further stage of the procedure. The fastener 600 must be simultaneously supported and rotated in order to be secured in hole 92. In the illustrated embodiment the fastener 600 is supported and attached to the bone by an endoscopic screwdriver apparatus 660, illustrated in FIGS. 28-29. The screwdriver 660 includes a proximal handle portion 662 (illustrated in dashed line), an elongated body portion 664, and a distal tool portion 666.

The distal tool portion 666, as illustrated in greater detail in FIG. 29 includes a substantially hexagonal outer periphery which is received in the substantially hexagonal recess 628 in the joint portion 614 of the screw member 602. A spring member at the distal tool portion 666 releasably engages the hexagonal recess 628 of the screw member 602 to support the fastener 600 during insertion and tightening. In the illustrated embodiment, a spring member 672 is configured to engage the side wall of the recess 628. More particularly, a channel/groove is provided in the tip portion 666 for receiving the spring member 672. The channel/groove includes a medial longitudinal notch portion 676, a proximal, angled channel portion 678, and a distal substantially transverse channel portion 680. The spring member 672 is preferably manufactured from stainless steel and has a medial portion 682 that is partially received in the longitudinal notch portion 676, an angled proximal portion 684 which is fixedly received in the angled channel portion 678, and a transverse distal portion 686 which is slidably received in the transverse channel 680. The medial portion 682 of the spring member 672 is partially exposed from the distal tip portion 666 and normally biased in a transverse outward direction with respect to the longitudinal axis (indicated by arrow E), in order to supply bearing force against the wall of the recess 628. Alternatively the distal tip portion of the screw driver may be magnetized in order to hold the screw portion 602. Similarly, the distal tip portion may include a ball bearing or similar member which is normally biased in a radially outward direction to engage the interior wall of the recess 628 to secure the fastener 600 to the screwdriver distal tip 666.

The insertion of the fastener 600 into the prepared hole 92 may be achieved by insertion of screwdriver 660 into conduit 20 (indicated by arrow G). This procedure may be visualized by the use of the endoscope 500 in conjunction with fluoroscopy. The screw portion 602 is threaded into the prepared hole 92 by the endoscopic screwdriver 660 (indicated by arrow. H). The endoscopic screwdriver 660 is subsequently separated from the fastener 600, by applying a force in the proximal direction, and thereby releasing the distal tip portion 666 from the hexagonal recess 628 (e.g., causing the transverse distal portion 686 of the spring member 672 to slide within the transverse recess 680 against the bias, indicated by arrow F), and removing the screwdriver 660 from the expandable conduit 20. An alternative method may use a guidewire, which is fixed in the hole 92, and a cannulated screw which has an internal lumen (as is known in the art) and is guided over the guidewire into the hole 92. The screwdriver would be cannulated as well to fit over the guidewire.

For a two-level fixation, it may be necessary to prepare several holes and attach several fasteners 600. Typically, the expandable conduit 20 will be sized in order to provide simultaneous access to all vertebrae in which the surgical procedure is being performed. In some cases, however, additional enlargement or repositioning of the distal portion of the expandable conduit may be required in order to have sufficient access to the outer vertebrae, e.g., the L4 and S1 vertebrae. In the illustrated embodiment, the expander apparatus 200 may be repeatedly inserted into the expandable conduit 20 and expanded in order to further open or position the skirt portion 24. In one procedure, additional fasteners are inserted in the L4 and S1 vertebrae in a similar fashion as the fastener 600 inserted in to the L5 vertebra as described above. (When discussed individually or collectively, a fastener and/or its individual components will be referred to by the reference number, e.g., fastener 600, housing 604, and all fasteners 600. However, when several fasteners and/or their components are discussed in relation to one another, an alphabetic subscript will be used, e.g., fastener 600a is moved towards fastener 600b.)

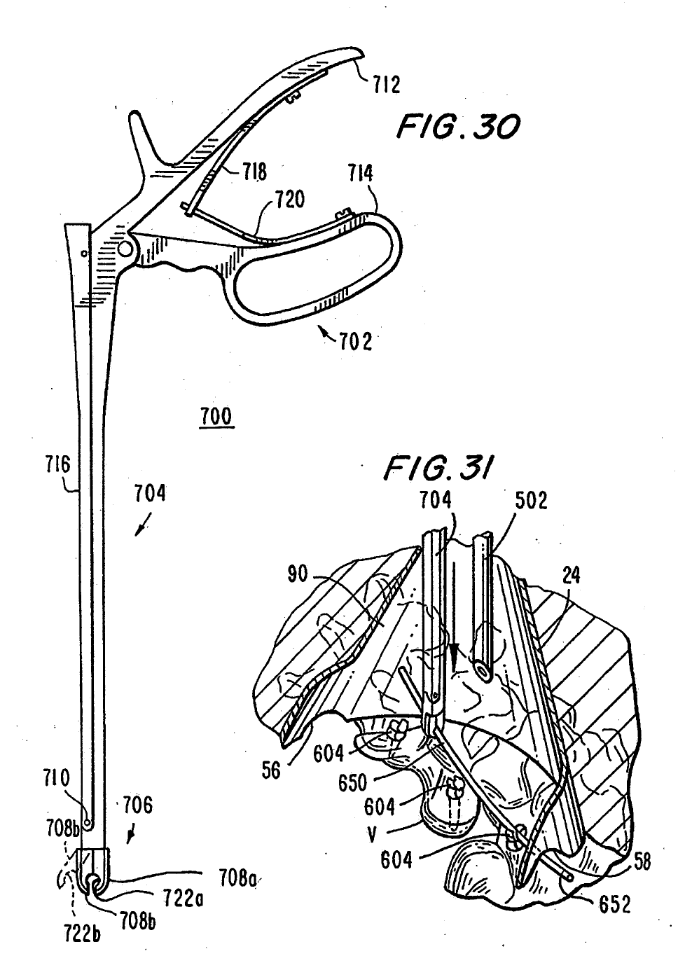

In a further stage of the procedure, the housing portions 604 of the fasteners 600 are substantially aligned such that their upright portions 630 and 631 face upward, and the notches 632 are substantially aligned to receive the elongated member 650 therein. The frictional mounting of the housing 604 to the screw member 602, described above, allows the housing 604 to be temporarily positioned until a subsequent tightening step, described below. Positioning of the housing portions 604 may be performed by the use of an elongated surgical instrument capable of contacting and moving the housing portion to the desired orientation. One such instrument for positioning the housings 604 is a grasper apparatus 700, illustrated in FIG. 30. The grasper apparatus 700 includes a proximal handle portion 702, an elongated body portion 704, and distal nose portion 706. The distal nose portion 706 includes a pair of grasping jaws 708a and 708b, which are pivotable about pin 710 by actuation of the proximal handle portion 702. The grasping jaws 708a and 708b are illustrated in the closed position in FIG. 30. As is known in the art, pivoting the movable handle 714 towards stationary handle 714 causes longitudinal movement of actuator 716, which in turn pivots the jaw 708b towards an open position (illustrated in dashed line). The biasing members 718 and 720 are provided to return the handles 712 and 714 to the open position and bias the jaws 708a and 708b to the closed position.

A subsequent stage in the process is the insertion of the elongated member 650 into the expandable conduit. The elongated member 650 is manufactured from a biocompatible material and must be sufficiently strong to maintain the positioning of the vertebrae, or other body structures. In the exemplary embodiment, the elongated members 650 are manufactured from Titanium 6/4 or titanium alloy. Alternatively, the elongated member 650 may be manufactured from stainless steel or other suitable material. The radii and length of the elongated members 650 are selected by the physician to provide the best fit for the positioning of the screw heads. Such selection may be performed by placing the elongated member 650 on the skin of the patient overlying the location of the fasteners and viewed fluoroscopically. For example, a 70 mm preformed rod having a 3.5″ bend radius may be selected for the spinal fixation.

The elongated member 650 is subsequently fixed to each of the fasteners 600, and more particularly, to the housings 604 of each fastener 600. The grasper apparatus 700, described above, is also particularly useful for inserting the elongated member 650 into the expandable conduit 20 and positioning it with respect to each housing 604. As illustrated in FIG. 30, the jaws 708a and 708b of the grasper apparatus 700 each has a curved contact portion 722a and 722b for contacting and holding the outer surface of the elongated member 650.

As illustrated in FIG. 31, the grasper apparatus 700 may be used to insert the elongated member 650 into the operative space 90 defined at least partially by the skirt portion 24 of the expandable conduit 20. The cut-out portions 56 and 58 provided in the skirt portion 24 assist in the process of installing the elongated member 650 with respect to the housings 604. The cut-out portions 56 and 58 allow an end portion 652 of the elongated member 650 to extend beyond the operative space without raising or repositioning the skirt portion 24. The elongated member 650 is positioned within the recesses in each housing 604 defined by grooves 632 disposed between upright members 630 and 631. The elongated member 650 is positioned in an orientation substantially transverse to the longitudinal axis of each housing 604.

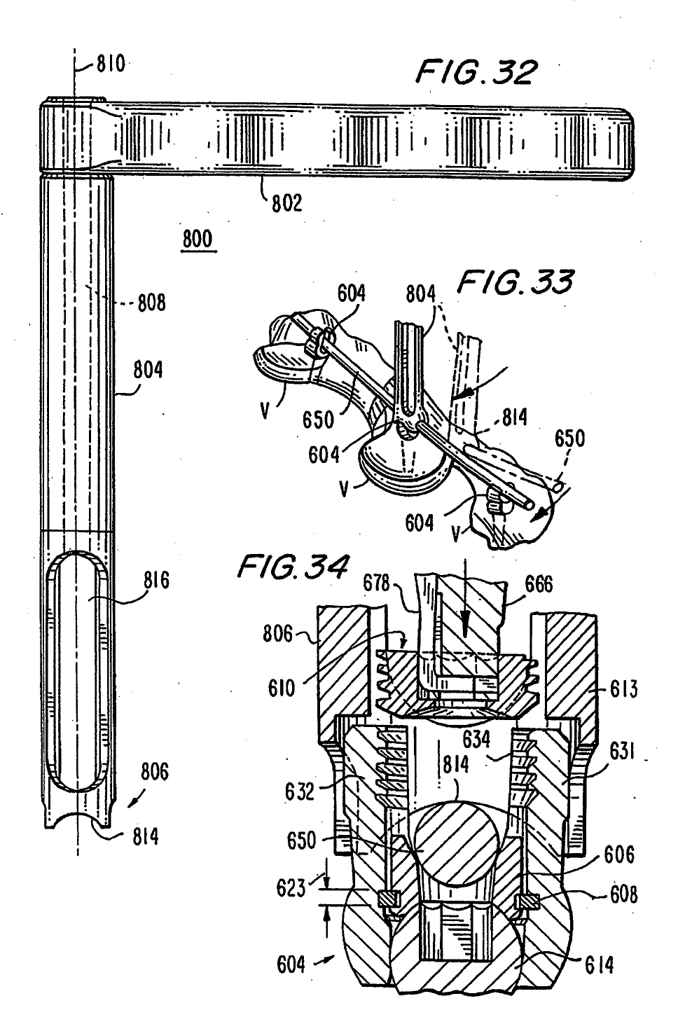

Further positioning of the elongated member 650 may be performed by guide apparatus 800, illustrated in FIG. 32. Guide apparatus 800 is useful in cooperation with an endoscopic screwdriver, such as endoscopic screwdriver 660 (illustrated in FIG. 28), in order to position the elongated member 650, and to introduce and tighten the cap screw 610, described above and illustrated in FIG. 27. Tightening of the cap screw 610 with respect to the housing 604 fixes the orientation of the housing 604 with respect to the screw portion 602 and fixes the position of the elongated member 650 with respect to the housing 604.

In the illustrated embodiment, the guide apparatus 800 has a proximal handle portion 802, an elongated body portion 804, and a distal tool portion 806. The elongated body portion 804 defines a central bore 808 (illustrated in dashed line) along its longitudinal axis 810. The central bore 808 is sized and configured to receive the endoscopic screwdriver 660 and cap screw 610 therethrough. In the exemplary embodiment, the diameter of the central bore 808 of the elongated body portion 804 is about 0.384-0.388 inches in diameter, and the external diameter of the endoscopic screwdriver 660 (FIG. 28) is about 0.25 inches. The proximal handle portion 802 extends transverse to the longitudinal axis 810, which allows the physician to adjust the guide apparatus 800 without interfering with the operation of the screwdriver 660.

The distal portion 806 of the apparatus includes several semicircular cut out portions 814 which assist in positioning the elongated member 650. As illustrated in FIG. 33, the cut out portions 814 are sized and configured to engage the surface of elongated member 650 and move the elongated member 650 from an initial location (illustrated in dashed line) to a desired location.

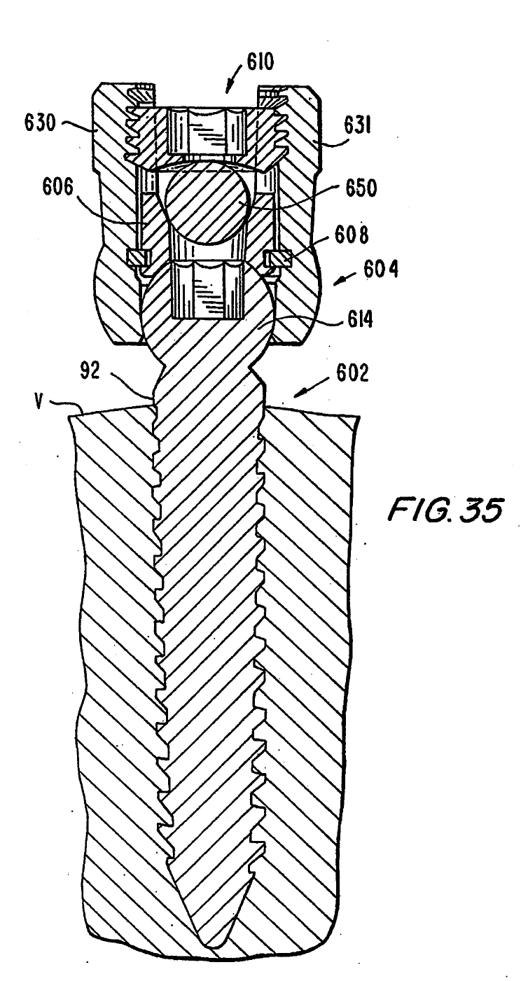

As illustrated in FIG. 34, the guide apparatus 800 is used in cooperation with the endoscopic screwdriver 660 to attach the cap screw 610. The distal end of the body portion 804 includes a pair of elongated openings 816, which permit the physician to endoscopically view the cap screw 610 retained at the distal tip 666 of the endoscopic screw driver 660.

The guide apparatus 800 and the endoscopic screwdriver 660 may cooperate as follows. The guide apparatus 800 is configured to be positioned in a surrounding configuration with the screwdriver 600. In the illustrated embodiment, the body portion 804 is configured for coaxial placement about the screwdriver 660 in order to distribute the contact force of the guide apparatus 800 on the elongated member 650. The distal portion 806 of the guide apparatus 800 may bear down on the elongated member 650 to seat the elongated member 650 in the notches 632 in the housing 604. The “distributed” force of the guide apparatus 800 may contact the elongated member 650 on at least one or more locations. In addition, the diameter of central bore 808 is selected to be marginally larger than the exterior diameter of cap screw 610, such that the cap screw 610 may freely slide down the central bore 808, while maintaining the orientation shown in FIG. 34. This configuration allows the physician to have effective control of the placement of the cap screw 610 into the housing 604. The cap screw 610 is releasably attached to the endoscopic screwdriver 660 by means of spring member 672 engaged to the interior wall of hexagonal recess 611 as it is inserted within the bore 808 of the body portion 804 of guide apparatus 800. The cap screw 610 is attached to the housing 604 by engaging the threads 615 of the cap screw 610 with the threads 634 of the housing.

As illustrated in FIG. 35, tightening of the cap screw 610 fixes the assembly of the housing 604 with respect to the elongated member 650. In particular, the distal surface of the cap screw 610 provides a distal force against the elongated member 650, which in turn drives the spacer member 606 against the joint portion 614 of the screw portion 602 , which is consequently fixed with respect to the housing 604.

If locations of the vertebrae are considered acceptable by the physician, then the fixation procedure is substantially complete once the cap screws 610 have been attached to the respective housings 604, and tightened to provide a fixed structure as between the elongated member 650 and the various fasteners 600. However, if compression or distraction of the vertebrae with respect to one another is required additional apparatus would be used to shift the vertebrae prior to final tightening all of the cap screws 610.

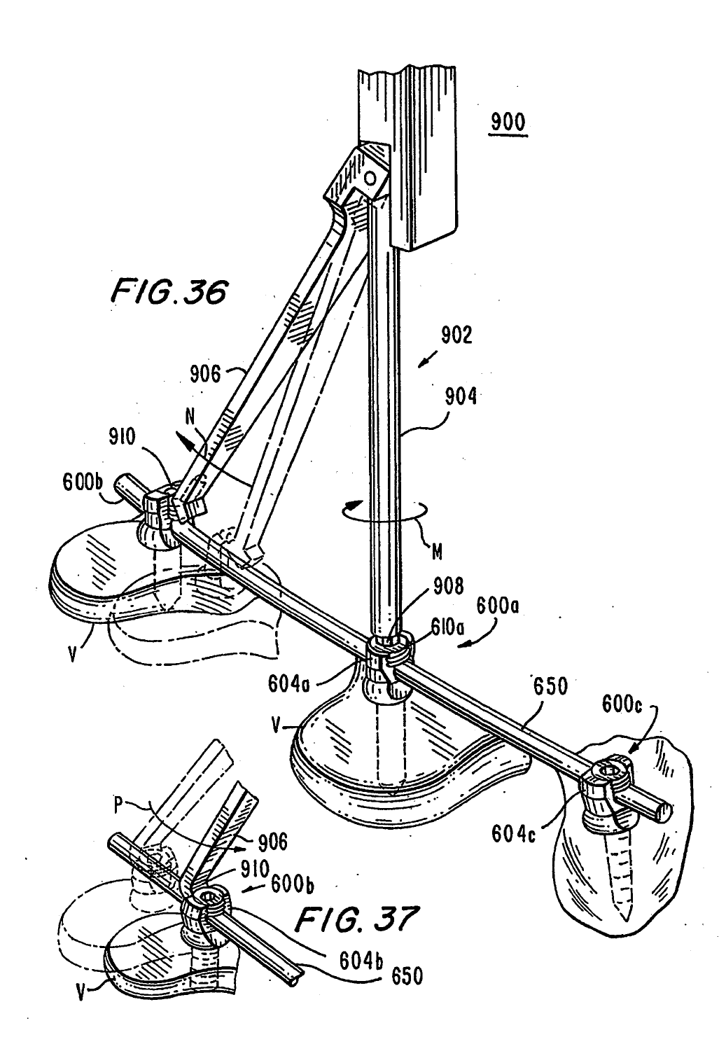

In the illustrated embodiment, this step is performed with a surgical instrument, such as compressor-distractor instrument 900, illustrated in FIG. 36, which is useful to relatively position bone structures in the cephcaudal direction and to fix their position with respect to one another. Thus, the compressor-distractor instrument 900 has the capability to engage two fasteners 600 and to space them apart while simultaneously tightening one of the fasteners to fix the spacing between the two vertebrae, or other bone structures. Moreover, the compressor-distractor instrument 900 may also be used to move two fasteners 600, and the vertebrae attached thereto into closer approximation and fix the spacing therebetween.

The distal tool portion 902 of the compressor-distractor instrument 900 is illustrated in FIG. 36. (Further details of the compressor-distractor apparatus is described in co-pending U.S. application Ser. No. 10/178,875, filed Jun. 24, 2002, entitled “Surgical Instrument for Moving Vertebrae,” which is hereby incorporated by reference herein in its entirety.) The distal tool portion 902 includes a driver portion 904 and a spacing member 906. The driver portion 904 has a distal end portion 908 with a plurality of wrenching flats configured to engage the recess 611 in the proximal face of the cap screw 610, and to apply torque to the cap screw. The driver portion 904 is rotatable about the longitudinal axis (indicated by arrow M) to rotate the cap screw 610 relative to the fastener 600. Accordingly, the driver portion 904 can be rotated to loosen the cap screw 610 on the fastener 600 and permit movement of the elongated member 650 connected with the vertebra relative to the fastener 600 connected with the vertebra. The cap screw 610 can also be rotated in order to tighten the cap screw 610 and clamp the elongated member 650 to the fastener 600.

The distal tool portion 902 may also include a spacing member, such as spacing member 906, which engages an adjacent fastener 600b while driver member 904 is engaged with the housing 604a to move the fastener 600b with respect to the fastener 600a. In the exemplary embodiment, spacing member 906 is a jaw portion which is pivotably mounted to move between a first position adjacent the driver portion and a second position spaced from the driver portion, as shown in FIG. 36. The distal tip 910 of the spacing member 906 is movable relative to the driver portion 904 in a direction extending transverse to the longitudinal axis.

As illustrated in FIG. 36, the spacer member 906 can be opened with respect to the driver portion 904 to space the vertebrae further apart (as indicated by arrow N). The distal portion 910 of the spacer member 906 engages the housing 604b of fastener 600b and moves fastener 600b further apart from fastener 600a to distract the vertebrae. Where the vertebrae are to be moved closer together, e.g. compressed, the spacer member 906 is closed with respect to the driver portion 904 (arrow P), as illustrated in FIG. 37. The distal portion 610 of spacer member 606 engages housing 604b of fastener 600b and moves fastener 600b towards fastener 600a. When the spacing of the vertebrae is acceptable to the physician, the cap screw 610a is tightened by the driver member 904, thereby fixing the relationship of the housing 604a with respect to elongated member 650, and thereby fixing the position of the vertebrae, or other bone structures, with respect to one another.

Once the elongated member 650 is fixed with respect to the fasteners 600, the procedure is substantially complete. The surgical instrumentation, such as the endoscope 500 is withdrawn from the surgical site. The expandable conduit 20 is also withdrawn from the site. The muscle and fascia typically close as the expandable conduit 20 is withdrawn through the dilated tissues in the reduced profile configuration. The fascia and skin incisions are closed in the typical manner, with sutures, etc. The procedure described above may be repeated for the other lateral side of the same vertebrae, if indicated.

II. Interbody Procedures that may be Preformed with the Above Apparatuses and Methods

Additional procedures that can be performed through an access device, e.g., an expandable conduit, may be combined with the procedures hereinbefore described. For example, the above procedures can be combined with a variety of interbody procedures, e.g., procedures that are performed at least in part in the space between adjacent vertebrae. As discussed above, an implant may be placed in the interbody space. Such implants are configured to foster bone growth in some embodiments between at least one surface thereof and at least one surface of at least one vertebra. In some embodiments, the implant is configured to preserve a degree of motion between the adjacent vertebrae. Preserving motion can reduce the likelihood that the patient will require additional procedures. Applying a motion preserving implant through an access device will reduce the complexity and the cost of the procedure, as well as the patient's postoperative pain and recovery time.

A. Apparatuses and Methods for Promoting Fusion of Adiacent Vertebrae

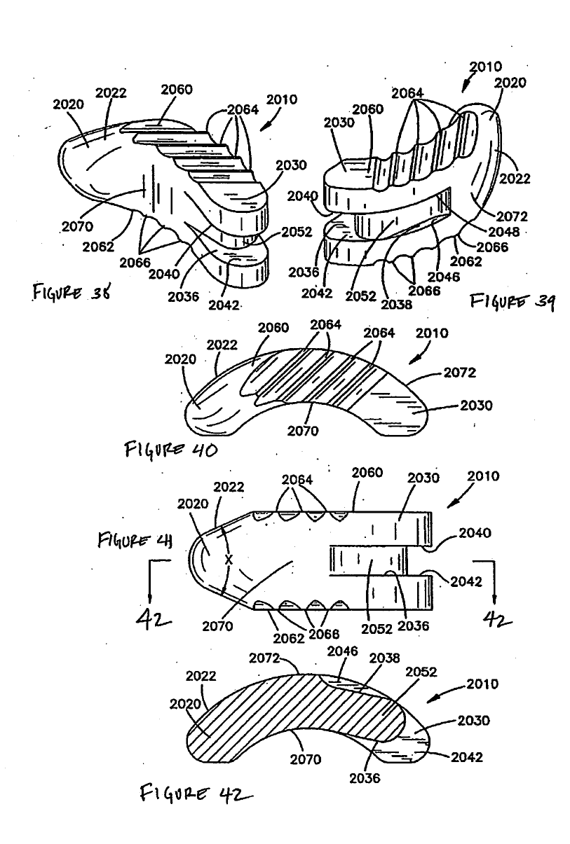

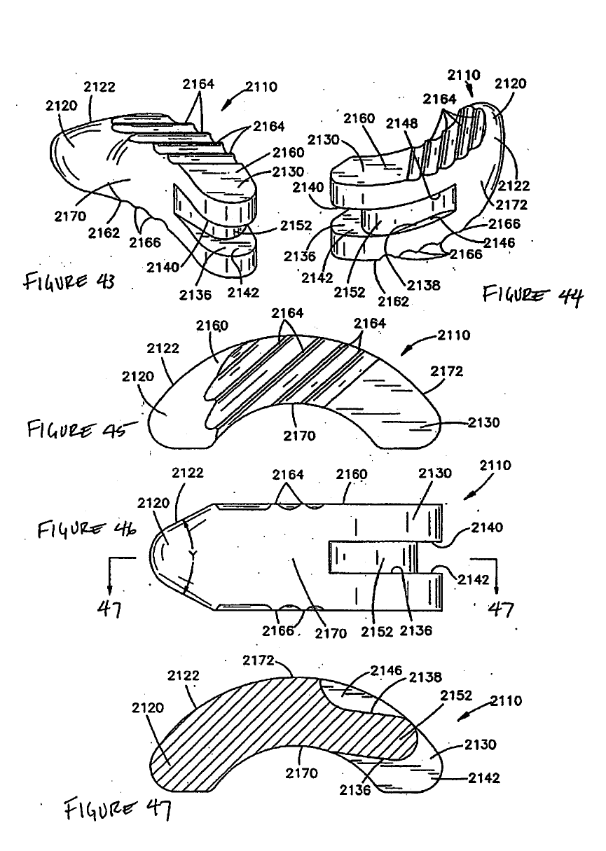

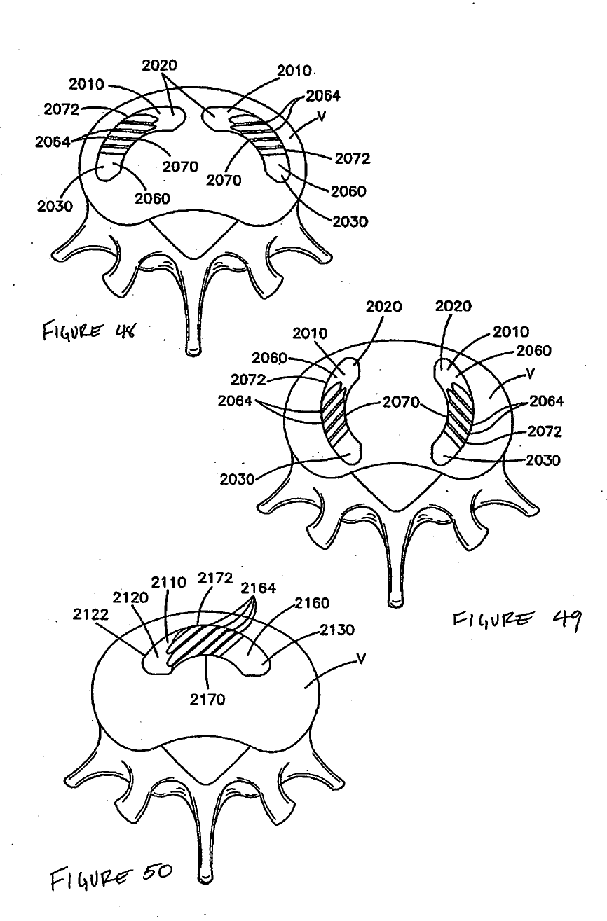

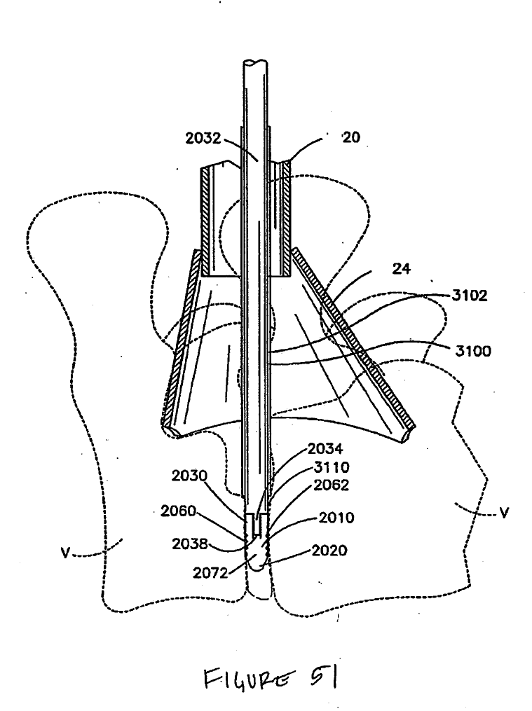

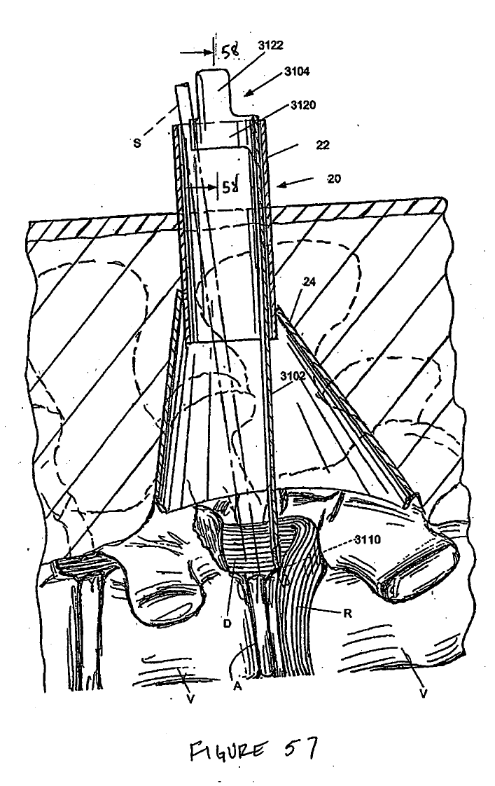

FIGS. 38-42 illustrate an embodiment of a fusion device or spinal implant 2010 that is inserted between the adjacent vertebrae. The spinal implant 2010 is placed between adjacent vertebrae to provide sufficient support to allow fusion of the adjacent vertebrae, as shown in FIGS. 48 and 49. The spinal implants 2010 are preferably made from an allograft material.

The spinal implant 2010 (FIGS. 38-42) has a first end 2020 for insertion between the adjacent vertebrae V. The first end 2020 has a tapered surface 2022 to facilitate insertion of the implant between the adjacent vertebrae V. The surface 2022 defines an angle X of approximately 45 degrees as shown in FIG. 41.

The spinal implant 2010 (FIGS. 38 and 39) has a second end 2030 that is engageable with a tool 2032 (FIG. 51) for inserting the implant between the adjacent vertebrae V. The tool 2032 has a pair of projections 2034, one of which is shown in FIG. 51, that extend into recesses 2036 and 2038 in the end 2030 of the implant 2010. The recesses 2036 and 2038 (FIGS. 38 and 39) extend from the second end 2030 toward the first end 2020. The recess 2036 (FIG. 41) is defined by an upper surface 2040 and a lower surface 2042 extending generally parallel to the upper surface 2040. The recess 2038 (FIG. 39) has a lower surface 2046 and an upper surface 2048 extending generally parallel to the lower surface 2046.

The recesses 2036 and 2038 define a gripping portion 2052. The projections 2034 on the tool 2032 extend into the recesses 2036 and 2038 and grip the gripping portion 2052. The projections 2034 engage the upper and lower surfaces 2040 and 2042 of the recess 2036 and the upper and lower surfaces 2046 and 2048 of the recess 2038. Accordingly, the tool 2032 grips the implant 2010 for inserting the implant between the adjacent vertebrae V.

The implant 2010 (FIGS. 38-41) has an upper surface 2060, as viewed in FIGS. 38-41, for engaging the upper vertebra V. The implant 2010 has a lower surface 2062, as viewed in FIGS. 38-41, for engaging the lower vertebra V. The upper and lower surfaces 2060 and 2062 extend from the first end 2020 to the second end 2030 of the implant 2010 and parallel to the upper and lower surfaces 2040, 2042, 2046, and 2048 of the recesses 2036 and 2038. The upper surface 2060 has teeth 2064 for engaging the upper vertebra V. The lower surface 2062 has teeth 2066 for engaging the lower vertebra V. Although FIGS. 38 and 39 show four teeth 2064 and four teeth 2066, it is contemplated that any number of teeth could be used.

A first side surface 2070 and a second side surface 2072 extend between the upper and lower surfaces 2060 and 2062. The first side surface 2070 extends along a first arc from the first end 2022 of the implant 2010 to the second end 2030. The second side surface 2072 extends along a second arc from the first end 2022 to the second end 2030. The first and second side surfaces 2070 and 2072 are concentric and define portions of concentric circles. The teeth 2064 and 2066 parallel to each other and extend between the side surfaces 2070 and 2072 and along secant lines of the concentric circles defined by the side surfaces.

The implant 2010 is formed by harvesting allograft material from a femur, as known in the art. The femur is axially cut to form cylindrical pieces of allograft material. The cylindrical pieces are then cut in half to form semi-cylindrical pieces of allograft material. The semi-cylindrical pieces of allograft material are machined into the spinal implants 2010.

A pair of spinal implants 2010 may be placed bilaterally between the adjacent vertebrae V. The expandable conduit 20 is inserted into the patient's body adjacent the vertebrae V. The skirt portion 24 of the expandable conduit 20 is radially expanded to provide a working space adjacent the vertebrae V. Disc material between the vertebrae V is removed using instruments such as kerrisons, rongeurs, or curettes. A microdebrider may also be utilized to remove the disc material. An osteotome, curettes, and scrapers are used to prepare end plates of the vertebrae V for fusion. Preferably, an annulus of the disc is left between the vertebrae V.

Distracters are used to sequentially distract the disc space until the desired distance between the vertebrae V is achieved. The fusion device or implant 2010 is placed between the vertebrae V using the tool 2032. The first end 2020 of the implant 2010 is inserted first between the vertebrae V. The implant 2010 is pushed between the vertebrae V until the end 2030 of the implant is between the vertebrae. A second spinal implant 2010 is inserted on the ipsilateral side using the same procedure.

A shield apparatus 3100 with an elongated portion 3102 may be used to facilitate insertion of the implants 2010 between the vertebrae V. A distal portion 3110 of the apparatus 3100 may be placed in an annulotomy. The implant 2010 is inserted with the side surface 2170 facing the elongated portion 3102 so that the apparatus 3100 can act as a “shoe horn” to facilitate or guide insertion of the implants 2010 between the vertebrae.

The implants 2010 may be inserted between the vertebrae V with the first ends 2020 located adjacent each other and the second ends 2030 spaced apart from each other, as shown in FIG. 48. The implants 2010 may also be inserted between the vertebrae V with the first ends 2020 of the implants 2010 spaced apart approximately the same distance that the second ends 2030 are spaced apart. It is contemplated that the implants 2010 may be inserted in any desired position between the vertebrae V. It is also contemplated that only one implant 2010 may be inserted between the vertebrae V. Furthermore, it is contemplated that the implants 2010 may be inserted between vertebrae using an open procedure.

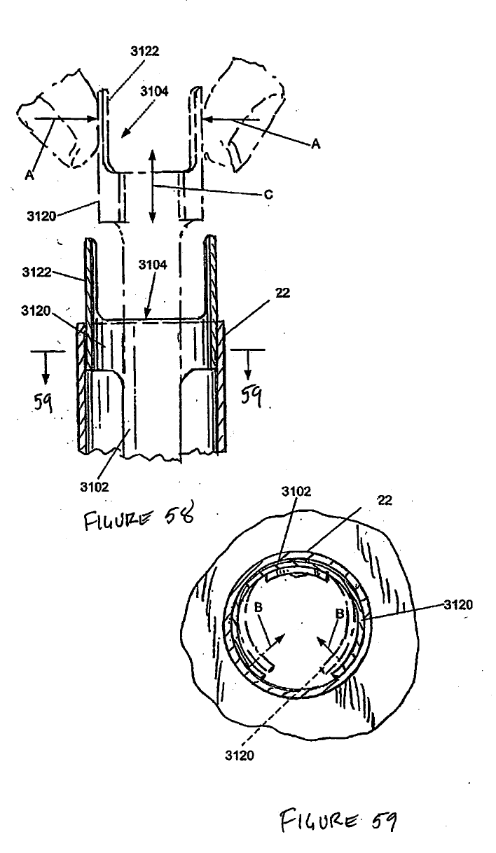

Another embodiment of a fusion device or spinal implant 2110 is illustrated in FIG. 43-47. The spinal implant 2110 is substantially similar to the embodiment disclosed in FIGS. 38-42. The implant 2110 is placed between the adjacent vertebrae V to provide sufficient support to allow fusion of the adjacent vertebrae, as shown in FIG. 50. The spinal implant 2110 is preferably made from an allograft material.

The spinal implant 2110 (FIGS. 41-45) has a first end 2120 for insertion between the adjacent vertebrae V. The first end 2120 has a tapered surface 2122 to facilitate insertion of the implant between the adjacent vertebrae V. The surface 2122 defines an angle Y of approximately 45 degrees as shown in FIG. 47.

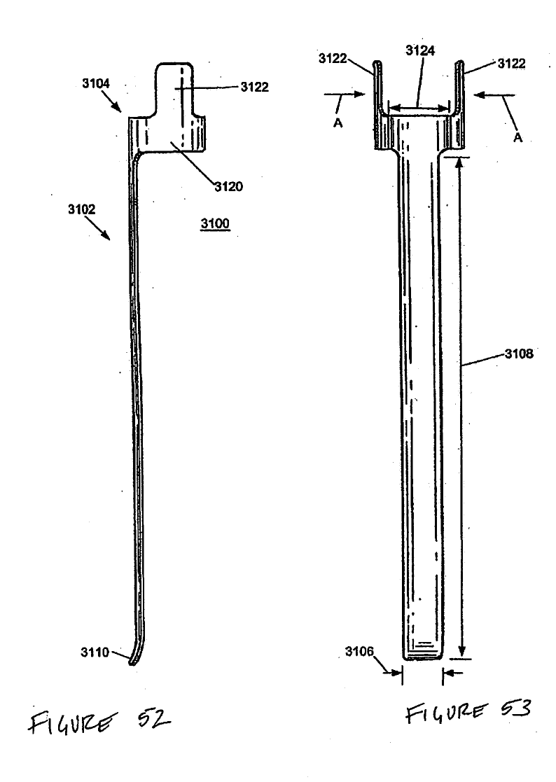

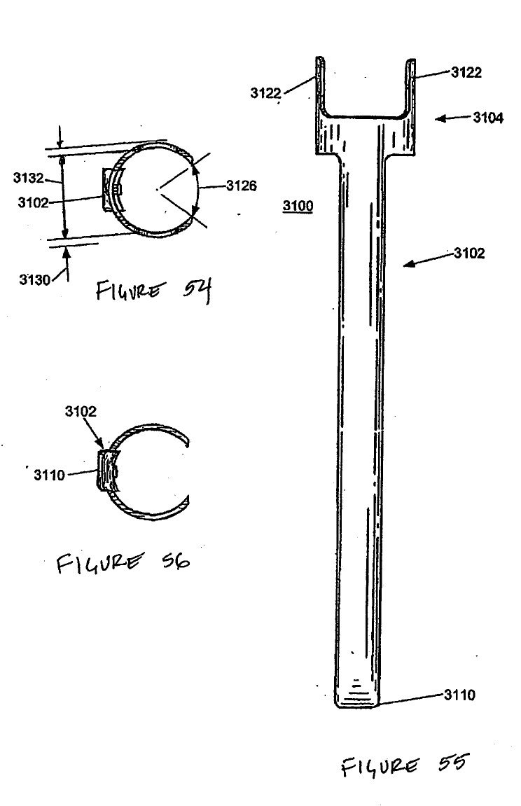

The spinal implant 2110 (FIGS. 43 and 44) has a second end 2130 that is engageable with the projections 2034 on the tool 2032 for inserting the implant between the adjacent vertebrae V. The projections 2034 extend into recesses 2136 and 2138 in the end 2130 of the implant 2110. The recesses 2136 and 2138 extend from the second end 2130 toward the first end 2120. The recess 2136 (FIGS. 43 and 46) is defined by an upper surface 2140 and a lower surface 2142 extending generally parallel to the upper surface 2140. The recess 2138 (FIG. 44) has a lower surface 2146 and an upper surface 2148 extending generally parallel to the lower surface 2146.

The recesses 2136 and 2138 define a gripping portion 2152. The projections 2034 on the tool 2032 extend into the recesses 2136 and 2138 and grip the gripping portion 2152. The projections 2034 engage the upper and lower surfaces 2140 and 2142 of the recess 2136 and the upper and lower surfaces 2146 and 2148 of the recess 2138. Accordingly, the tool 2032 grips the implant 2110 for inserting the implant between the adjacent vertebrae V.

The implant 2110 (FIGS. 43-47) has an upper surface 2160, as viewed in FIGS. 43-47, for engaging the upper vertebra V. The implant 2110 has a lower surface 2162, as viewed in FIGS. 43-47, for engaging the lower vertebra V. The upper and lower surfaces 2160 and 2162 extend from the first end 2120 to the second end 2130 of the implant 2110 and parallel to the upper and lower surfaces 2140, 2142, 2146, and 2148 of the recesses 2136 and 2138. The upper surface 2160 has teeth 2164 for engaging the upper vertebra V. The lower surface 2162 has teeth 2166 for engaging the lower vertebra V. Although FIG. 44 shows four teeth 2164 and four teeth 2166, it is contemplated that any number of teeth could be used.

A first side surface 2170 and a second side surface 2172 extend between the upper and lower surfaces 2160 and 2162. The first side surface 2170 extends along a first arc from the first end 2122 of the implant 2110 to the second end 2130. The second side surface 2172 extends along a second arc from the first end 2120 to the second end 2130. The first and second side surfaces 2170 and 2172 are concentric and define portions of concentric circles. The teeth 2164 and 2166 extend parallel to each other, and between the side surfaces 2170 and 2172 along secant lines of the concentric circles defined by the side surfaces.

The implant 2110 is formed by harvesting allograft material from a femur, as is known in the art. The femur is axially cut to form cylindrical pieces of allograft material. The cylindrical pieces are then cut in half to form semi-cylindrical pieces of allograft material. The semi-cylindrical pieces of allograft material are machined into the spinal implants 2110.

A spinal implant 2110 is placed unilaterally between the adjacent vertebrae V. The expandable conduit 20 is inserted into the patient's body adjacent the vertebrae V. The skirt portion 24 of the expandable conduit 20 is radially expanded to provide a working space adjacent the vertebrae V. Disc material between the vertebrae V is removed using instruments such as kerrisons, rongeurs, or curettes. A microdebrider may also be utilized to remove the disc material. An osteotome, curettes, and scrapers are used to prepare end plates of the vertebrae V for fusion. Preferably, an annulus of the disc is left between the vertebrae V.

Distracters are used to sequentially distract the disc space until the desired distance between the vertebrae V is achieved. The implant 2110 is placed between the vertebrae V using the tool 2032. It is contemplated that the apparatus 3100 could be used also. The first end 2120 of the implant 2110 is inserted first between the vertebrae V. The implant 2110 is pushed between the vertebrae V until the end 2130 of the implant is between the vertebrae. It is contemplated that the implant 2110 may be inserted in any desired position between the vertebrae V. It is also contemplated that more than one implant 2110 may be inserted between the vertebrae.

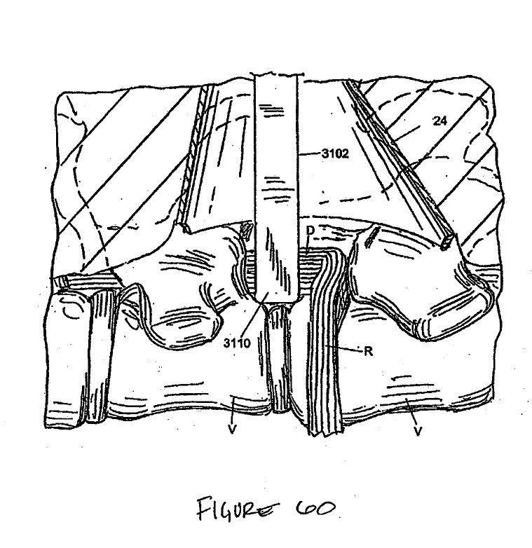

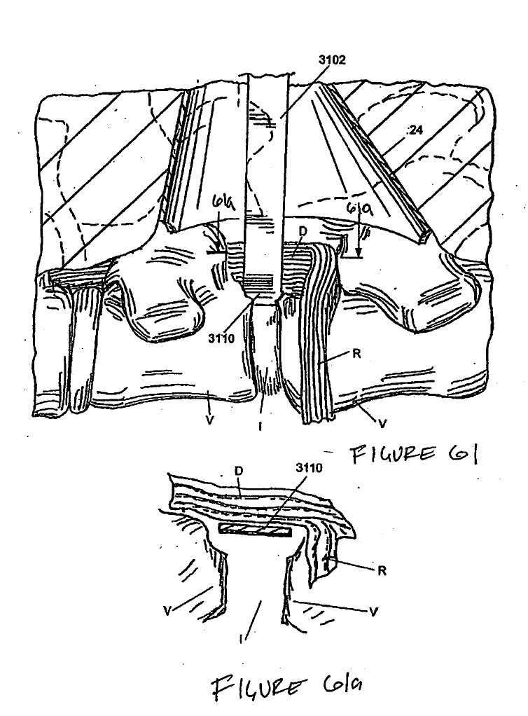

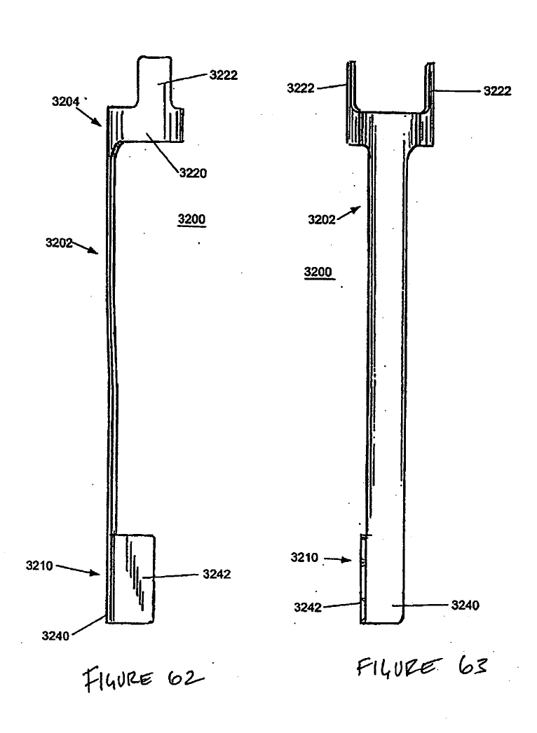

The apparatus or shield 3100 for use in placing the fusion devices or spinal implants between the vertebrae is illustrated in FIGS. 52-56. The apparatus 3100 includes an elongated body portion 3102, which protects the nerve root or dura, and a mounting portion 3104, which allows for the surgeon to releasably mount the apparatus 3100 to the expandable conduit 20. Consequently, the surgeon is able to perform the surgical procedures without requiring the surgeon or an assistant to continue to support the apparatus 3100 throughout the procedure, and without reducing the field of view.

The apparatus 3100 may be manufactured from a biocompatible material such as, but not limited to, stainless steel. In the exemplary embodiment, apparatus 3100 is manufactured from stainless steel having a thickness of about 0.02 inches to about 0.036 inches. The elongated body portion 3102 has dimensions which correspond to the depth in the body in which the procedure is being performed, and to the size of the body structure which is to be shielded by elongated body portion 3102. In the exemplary embodiment, the elongated body portion 3102 has a width 3106 of about 0.346 inches and a length of about 5.06 inches (FIG. 53), although other dimensions would be appropriate for spinal surgical procedures performed at different locations, or for surgical procedures involving different body structures. The distal tip portion 3110 of the apparatus 3100 may have a slightly curved “bell mouth” configuration which allows for atraumatic contact with a body structure, such as a nerve. It is contemplated that the elongated body portion may have any desired shape.

The mounting portion 3104 allows the apparatus 3100 to be secured to a support structure in any number of ways. In the exemplary embodiment, mounting portion 3104 may include a ring portion. As seen in FIGS. 53, 54, and 56, ring portion 3120 has a substantially ring-shaped configuration with an opening 3124, which defines an angle 3126 of about 90 degrees of the total circumference of the ring portion 3120. As will be described in greater detail below, the angle 3126 is a nominal value, because the ring portion 3104 is resilient, which permits the opening 3124 to change size during the mounting process.

In the illustrated embodiment, the mounting portion 3104 has a substantially cylindrical configuration in order to be mounted within the interior lumen of the expandable conduit 20, as will be described below. The ring portion 3104 has an exterior dimension 3130 of about 0.79 inches, and an interior dimension 3132 of about 0.76 inches. It is understood that the dimensions of the ring portion 3104 would be different if the expandable conduit 20 has a different interior dimension. Moreover, the cylindrical shape of the ring portion 3104 would change if the apparatus 3100 is used with a support member having a differently shaped internal lumen.

Finger grip portions 3122 extend from the mounting portion 3104 and allow the surgeon to apply an inwardly directed force (as indicated by arrows A) to the ring portion 3120. The resilient characteristics of the ring portion 3120 allow the material to deflect thereby reducing the exterior dimension 3130 and reducing the spacing 3124. Releasing the finger grip portions 3122 allows the ring portion to move towards its undeflected condition, thereby engaging the interior wall of the expandable conduit 20.

The elongated body portion 3102 and the mounting portion 3104 may be. manufactured from a single component, such as a sheet of stainless steel, and then the mounting portion 3104 may be subsequently formed into a substantially cylindrical shape. In another embodiment, the mounting portion 3104 may be manufactured as a separate component and attached to the elongated body portion, by techniques such as, but not limited to welding and securement by fasteners, such as rivets.

The expandable conduit 20 serves as a stable mounting structure for apparatus 3100. In particular, mounting portion 3104 is releasably mounted to the interior wall of proximal wall portion 22 of expandable conduit 20. Elongated body portion 3102 extends distally into the operative site to protect the desired body structure, such as the nerve, as will be described below.

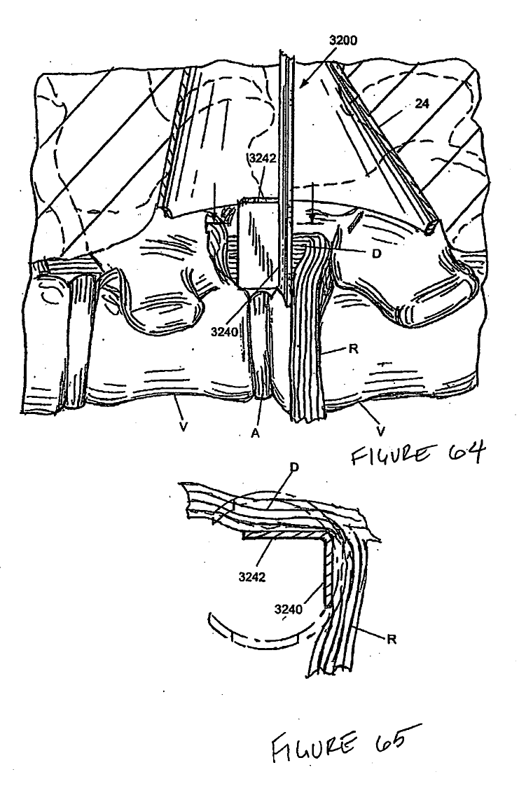

To install the apparatus 3100 within the interior passage of the proximal wall portion 22, the surgeon may apply an inwardly directed force on the ring portion 3120, thereby causing the ring portion to resiliently deform, as illustrated by dashed line and arrows B in FIGS. 58-59. The surgeon subsequently inserts the apparatus 3100 into the interior lumen of the proximal wall portion 22 (as indicated by arrow C) to the position of ring portion 3104 illustrated in solid line in FIGS. 58-59. When the surgeon releases the finger grip portions 3122, the ring portion 3120 resiliently moves towards its undeflected configuration, thereby engaging the interior lumen of the proximal wall portion 22. The mounting portion 3104 described herein has the advantage that it is easily removed and/or moved with respect to the conduit 20 without disturbing the position of the conduit 20 or any other instrumentation.

As illustrated in FIGS. 57 and 59, the configuration of the mounting portion 3104 and the elongated body portion 3102 allow the elongated body portion to occupy a small space along the periphery of the proximal wall portion 3122. This allows the apparatus to protect the desired body structure without blocking access for the insertion of other surgical instrumentation, and without blocking visibility by the surgeon during the procedure.

The mounting portion 3104 is one exemplary configuration for mounting the apparatus 3100 to the support structure. It is contemplated that the apparatus 3100 may be-mounted within the expandable conduit in another manner.

When in position, the distal end portion 3110 covers the exiting nerve root R, while exposing the disc amiulus A (See FIG. 57). As discussed above, the debridement and decortication of tissue covering the vertebrae, as well as a facetecomy and/or laminectomy if indicated, are performed prior to the insertion of apparatus 3100 into the surgical space. Thus, there is no need to displace or retract tissue, and apparatus 3100 merely covers the nerve root and does not substantially displace the nerve root or any other body tissue. It is understood that term “cover” as used herein refers to apparatus 3100 being a small distance adjacent to the body structure, or in contact with the body structure without applying significant tension or displacement force to the body structure.

Additional surgical instrumentation S may be inserted into the expandable conduit to perform procedures on the surrounding tissue. For example, an annulotomy may be performed using a long handled knife and kerrisons. A discectomy may be completed by using curettes and rongeurs. Removal of osteophytes which may have accumulated between the vertebrae may be performed using osteotomes and chisels.

As illustrated in FIG. 60, the elongated body portion 3102 is rotated to protect the spinal cord, or dura D, during the above procedures. The surgeon may change the position of the apparatus 3100 by approximating the finger grips 3122 to release the ring portion from engagement with the inner wall of the proximal wall portion 22, and then re-position the apparatus 3100 without disturbing the expandable conduit 20 (as shown in FIG. 58).

During certain surgical procedures, it may be useful to introduce crushed bone fragments or the fusion devices 2010 or 2110 to promote bone fusion. As illustrated in FIGS. 61-61a, apparatus 3100 is useful to direct the implants into the interbody space I between adjacent vertebrae V. As shown in the figures, the distal portion 3110 of the elongated body portion 3102 is partially inserted into the interbody space I. The distal end portion 3110, is positioned between adjacent vertebrae V, and creates a partially enclosed space for receiving the implants or other material therein.

Another embodiment of the apparatus or shield is illustrated in FIGS. 62-63, and designated apparatus 3200. Apparatus 3200 is substantially identical to apparatus 3100, described above, with the following differences noted herein. In particular, distal end portion 3210 includes a pair of surfaces 3240 and 3242. Surface 3240 is an extension of elongated shield portion 3202, and surface 3242 extends at an angle with respect to surface 3240. In the exemplary embodiment, surfaces 3240 and 3242 defined an angle of about 90 degrees between them. Alternatively another angle between surfaces 3240 and 3242 may be defined as indicated by the body structures to be protected.

As illustrated in FIGS. 64-65, distal end portion 3210 allows the apparatus to provide simultaneous shielding of both the dura D and the nerve root R. In FIGS. 64-65, surface 3242 shields the dura D, and surface 3240 shields the nerve root R. It is understood that surfaces 3240 and 3242 may be interchanged with respect to which tissue they protect during the surgical procedure.

After the spinal implants 2010 or 2110 are inserted between the vertebrae V, the fasteners 600 may be attached to the vertebrae. Prior to attachment of the fasteners, the location of the fastener attachment is confirmed. In the exemplary embodiment, the pedicle entry point of the L5 vertebra is located using visual landmarks as well as lateral and A/P fluoroscopy, as is known in the art. With reference to FIG. 25, the entry point 92 is prepared with an awl 550. A pedicle hole is completed at the entry point 92 using instruments known in the art such as a straight bone probe, a tap, and a sounder. The sounder, as is known in the art, determines whether the hole that is made is surrounded by bone on all sides, and that there has been no perforation of the pedicle wall.

After the pedicle hole at the entry point 92 is provided (or at any point during the procedure), an optional step is to adjust the location of the skirt portion 24 of the expandable conduit 20. This may be performed by inserting the expander apparatus 200 into the expandable conduit 20, expanding the distal portions 210, and contacting the inner wall of the skirt portion 24 to move the skirt portion 24 to the desired location. This step may be performed while the endoscope 500 is positioned within the expandable conduit 20, and without substantially disturbing the location of the proximal portion of the expandable conduit 20 to which the endoscope mount platform 300 may be attached.

B. Apparatuses and Methods for Replacing a Nucleus Pulposus and Preserving Motion

Another type of procedure that can be performed by way of the systems and apparatuses described herein involves replacement of one or more of a patient's nucleus pulposi with a replacement disc nucleus, e.g., a prosthetic device, that provides the functions of the natural nucleus pulposus while preserving or restoring a degree of normal motion after recovery. A variety of replacement disc nuclei that may be applied to replace a damaged or degenerating nucleus are described below. The access devices and systems described herein enable these devices and methods associated therewith to be practiced minimally invasively.

1. Replacement Disc Nucleus Comprising a Pliable Enclosure

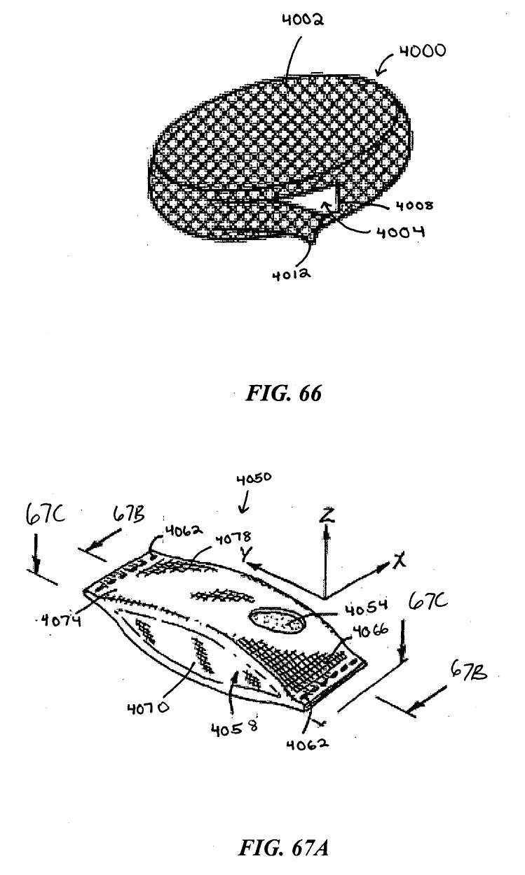

FIG. 66 shows a first embodiment of a replacement disc nucleus 4000 that comprises a pliable enclosure 4002. As used herein, the term “enclosure” is a broad term and is used in its ordinary sense and includes a structure within which a volume may be at least partially defined. In one embodiment, the enclosure 4002 formed of a porous material that permits body fluids to diffuse therethrough. The enclosure 4002 may be formed as a bag, a sac, a frame-like structure, or any other suitable arrangement.

The enclosure 4002 preferably defines a volume 4004 which may be increased and/or decreased during application to a patient's spine. For example, the enclosure 4002 is capable of having a first configuration prior to insertion into an intervertebral disc space, wherein the volume 4004 is relatively small and a second configuration after inserted into a patient, wherein the volume 4004 is relatively large. The enclosure 4002 may be compressed prior to insertion, and then expanded (or allowed to expand) during or after insertion.. In one application, the enclosure 4002 is compressed prior to insertion into an intervertebral disc space and is expanded (or permitted to expand) before a filler medium is advanced into the volume 4004 defined by the enclosure 4002.

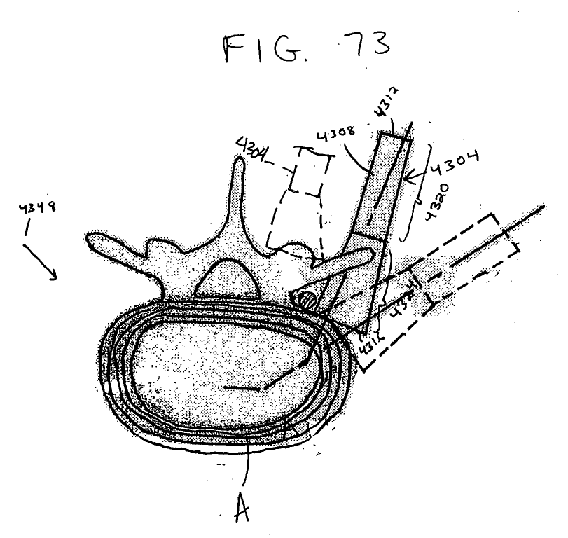

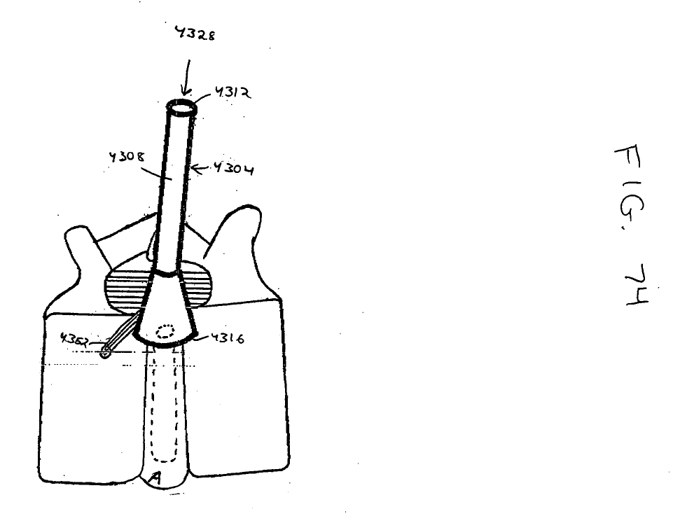

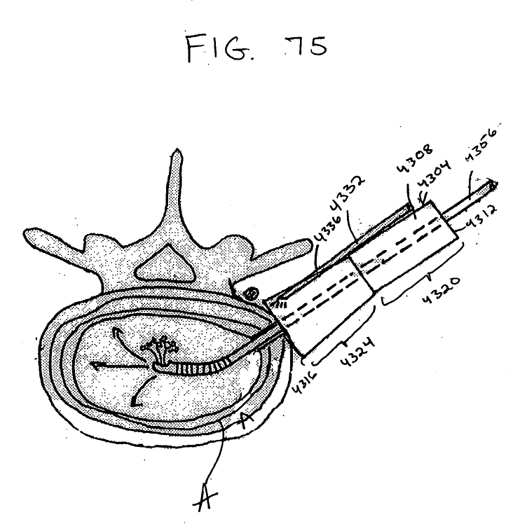

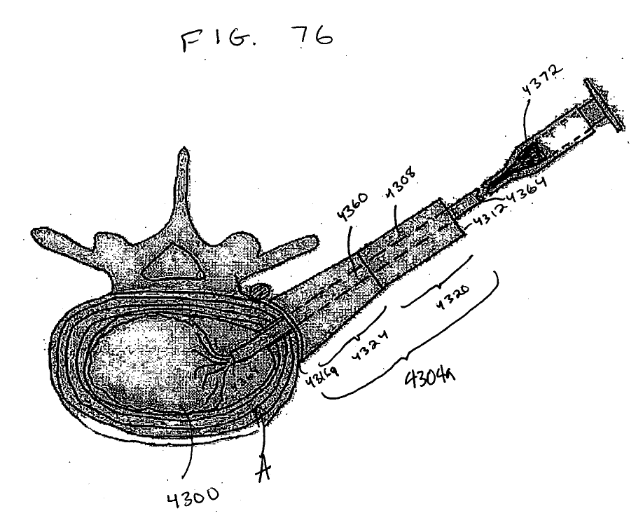

In one application, an expandable member is delivered into the volume 4004, which had previously been reduced in size, e.g., by compressing the enclosure 4002. The expandable member is expanded to expand the enclosure 4002 to increase the size of the volume 4004 before the filler medium is delivered. In one embodiment, the enclosure 4002 includes a balloon or bladder configured to facilitate the expansion of the enclosure 4002 from the compressed state to the expanded state. In one application, the balloon or bladder is filled with a suitable fluid (e.g., liquid or gas) to inflate the balloon or bladder and thereby to expand the enclosure 4002. These and other methods related to the enclosure 4002 are discussed in greater detail below in connection with FIGS. 73-78.

The enclosure 4002 preferably includes an aperture 4008 that may be opened and closed as needed. In the illustrated embodiment, the aperture 4008 is formed by retracting a flap 4012 or other similar structure. In another embodiment, a slit may be provided in addition to or in place of the flap 4012. In one embodiment, the flap 4012 can be securely closed so that the filler medium generally is contained within the volume 4004. Secure closure of the flap 4012 may be achieved by suturing the flap 4012 closed or by providing some other closure mechanism or device between the flap 4012 and the adjacent portion of the enclosure 4002.

Where the enclosure 4002 is configured to be filled by a filler medium, the filler medium may be any suitable medium, e.g., morselized nucleus pulposus from the patient, allograft material, or other biocompatible materials. The filler medium may also be allograft nucleus pulposus, xenograft nucleus pulposus, other tissue and/or synthetic materials such hydrogels. In one application, the nucleus material removed prior to insertion of the enclosure 4002 is ground up, e.g., morselized, and placed inside the enclosure 4002 to expand the enclosure 4002.

Various techniques may be performed to prevent the enclosure 4002 or filler medium from migrating from the position in which the enclosure 4002 and filler medium are originally placed. For example, one or both of the filler medium or the enclosure 4002 may be configured to encourage ingrowth of bone between an adjacent vertebra and the replacement disc nucleus 4000. In another arrangement, the filler medium is physically coupled with, e.g., woven or stapled, into the enclosure 4002 to deter migration of the inflation medium from the volume 4004. In another embodiment, the enclosure 4002 is configured to receive a suture or other structure of device, e.g., a staple, configured to couple with enclosure 4002 with one or more anatomical aspect, such as an inside surface of a disc annulus.

In one embodiment, the enclosure 4002 is or contains a self-expanding member. In application, the self-expanding enclosure may be delivered in a compressed configuration, as discussed above, and then released and permitted to expand within an intervertebral disc space. The self-expanding enclosure may include one or more spring-like hoops separated by an elastic material, such as rubber or silicone. The enclosure, or a portion of the material separating the hoops of the enclosure, could also include a shape memory material that enables the enclosure to change from a shape with an aperture (to allow insertion of a filler medium) to a shape with a small slit (to close the aperture).

In one application, as discussed more fully below in connection with FIGS. 73-77, an aperture may be formed in an annulus of a natural disc, providing a door-like flap in the annulus tissue. The aperture in the annulus may be configured such that the enclosure 4002 in the collapsed or compressed state may pass therethrough. The enclosure 4002 is then placed inside the intervertebral space, and actuated to the expanded state in any suitable manner. In some applications, fasteners such as sutures, staples, and so forth, may be inserted through the aperture 4008 into the volume 4004 and through at least a portion of, e.g., a wall of, the enclosure 4002 and into an anatomical structure, such as disc tissue, e.g., annulus tissue.

Further details relating to replacement disc nuclei having pliable enclosures may be found in U.S. patent application Ser. No. 10/120,763 filed on Apr. 11, 2002, and published as Publication No. 2002/0165542 on Nov. 7, 2002, which is hereby incorporated herein by reference in its entirety.

2. Replacement Disc Nucleus Including a Hydrogel

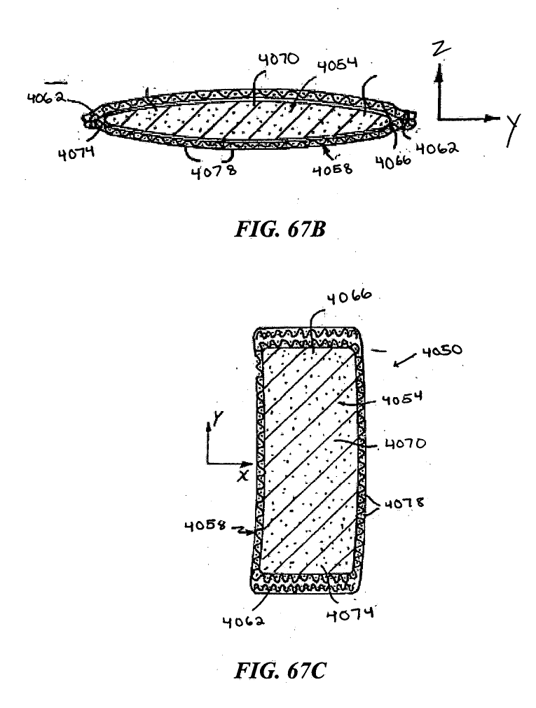

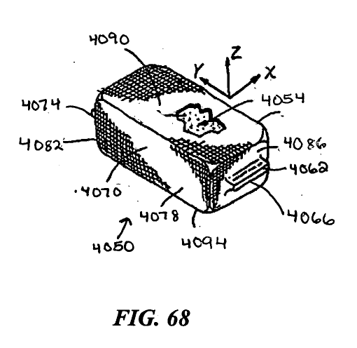

FIGS. 67A-68 illustrate another embodiment of a replacement disc nucleus 4050 that includes a hydrogel. In one embodiment, the replacement disc nucleus 4050 includes a hydrogel core 4054, and a constraining jacket 4058. The constraining jacket 4058 is secured about the hydrogel core 4054 by closures 4062 located at opposite ends of the constraining jacket 4058.

The replacement disc nucleus 4050 is described below as having a first, pre-replacement disc nucleus shape and a second, post-replacement disc nucleus shape. To this end, because the hydrogel core 4054 is dehydrated prior to implant and hydrated following implant, the pre-implant shape can also be referred to as a dehydrated shape; whereas the post-implant shape is referred to as a hydrated shape. As a point of reference, FIG. 67A depicts the dehydrated shape; whereas FIG. 68 depicts the hydrated shape.

In one embodiment, the hydrogel core 4054 is configured to imbibe fluids, expanding from a dehydrated state (shown in FIG. 67A) to a hydrated state (FIG. 68). In this regard, the hydrogel core 4054 is formulated as a mixture of hydrogel polyacrylonitrile in one embodiment. In particular, acrylamide and acrylonitrile (block co-polymer) are used in one embodiment. Alternatively, the hydrogel core 4054 can be any hydrophilic acrylate derivative with a unique multi-block co-polymer structure or any other hydrogel material having the ability to deform and reform in a desired fashion in response to placement and removal of loads, such as a keratin-derived hydrogel. Even further, a biologically safe polymer that can imbibe fluids while maintaining its structure under various stresses is acceptable. For example, the hydrogel core 4054 can be formulated as a mixture of polyvinyl alcohol and water. Much like a natural nucleus, the hydrogel core 4054 will initially swell from a dehydrated state as it absorbs fluid. When hydrated, the hydrogel core 4054 will have a water content of 25-90 percent in one embodiment. The hydrogel material used for the hydrogel core 4054 in the first embodiment is manufactured under the trade name HYPAN® by Hymedix International, Inc. of Dayton, N.J.

As shown in FIG. 67A, the hydrogel core 4054 defines a leading end 4066, a central portion 4070 and a trailing end 4074. As described in greater detail below, the leading end 4066 and the trailing end 4074 are in reference to a preferred orientation of the replacement disc nucleus 4050 during an implantation procedure. For the purposes of this disclosure, directional terminology, such as “leading” and “trailing,” are with reference to one possible orientation of the replacement disc nucleus 4050 during implantation. It should be understood, however, the replacement disc nucleus 4050 can be orientated in any direction relative to a nucleus cavity, also referred to herein as an interbody space. As such, the directional terms are provided for purposes of illustration only.

As a point of reference, the replacement disc nucleus 4050 is defined by a width (x-axis in FIG. 67A), a length (y-axis in FIG. 67A) and a height (z axis in FIG. 67A). With this in mind, the hydrogel core 4054, and thus the replacement disc nucleus 4050, is fabricated to assume a streamlined shape in the dehydrated state. The term “streamlined” is with reference to the hydrogel core 4054 being configured, in the dehydrated state, to taper or decrease in height (z-axis) from the central portion 4070 to the leading end 4066. In one embodiment, in the dehydrated state, the hydrogel core 4054 is further configured to taper or decrease in height (z-axis) from the central portion 4070 to the trailing end 4074. With this preferred embodiment, then, opposing sides of the hydrogel core 4054 are generally convex, resulting in the generally convexo-convex shape. While the taper or decrease in height (z-axis) is preferably uniform, other designs are acceptable. The “streamlined” shape in the dehydrated state relates to the central portion 4070 tapering in height to the leading end 4066. Further, in one embodiment, the central portion 4070 also tapers in height to the trailing end 4074.

In addition to the above-described streamlined shape, in one embodiment, a top, cross-sectional view shows the central portion 4070 of the hydrogel core 4054 as being curved. More particularly, opposing sides of the hydrogel core 4054 curve in a generally symmetrical fashion from the leading end 4066 to the trailing end 4074. Alternatively, the opposing side may be linear, non-symmetrical etc.

Completely surrounding the hydrogel core 4054 is the constraining jacket 4058. The constraining jacket 4058 is preferably a flexible tube made of tightly woven high molecular weight, high tenacity polymeric fabric. In one embodiment, high molecular weight polyethylene is used as the weave material for the constraining jacket 4058. However, polyester or any high tenacity polymeric material can be employed, and carbon fiber yarns, ceramic fibers, metallic fibers, etc., also are acceptable.

The constraining jacket 4058 is preferably made of fibers that have been highly orientated along their length. As a result, the constraining jacket 4058 material, while flexible, has little elasticity or stretch. The constraining jacket 4058 defines a generally fixed maximum volume, including a generally fixed length (y-axis of FIG. 67A). In one embodiment, the generally fixed maximum volume of the constraining jacket 4058 is less than a theoretical volume of the hydrogel core 4054 if allowed to completely hydrate without constraint. Thus, because the hydrogel core 4054 has a natural, fully hydrated volume greater than the constraining jacket 4058, the constraining jacket 4058 will be tight about the hydrogel core 4054 when hydrated, as described in greater detail below. Additionally, the volume differential between the constraining jacket 4058 and the hydrated hydrogel core 4054 serves to extend the useful life of the replacement disc nucleus 4050. In particular, the constraining jacket 4058 effectively prevents the hydrogel core 4054 from reaching its natural hydration level. Consequently, the hydrogel core 4054 will have a generally constant affinity for imbibing additional fluid. Finally, the hydrogel core 4054 is preferably configured such that in the dehydrated state, the hydrogel core 4054 has a length approximating the generally fixed maximum length of the constraining jacket 4058. Thus, the hydrogel core 4054 causes the constraining jacket 4058 to be relatively taut along its length (y-axis). Notably, the hydrogel core 4054 in the dehydrated state does not encompass the entire available volume of the constraining jacket 4058.

In one embodiment, the preferred woven construction of the constraining jacket 4058 creates a plurality of small openings 4078. Each of the plurality of small openings 4078 preferably is large enough to allow bodily fluids to interact with the hydrogel core 4054 otherwise maintained within the constraining jacket 4058. However, each of the plurality of small openings 4078 preferably is small enough to prevent most if not all of the hydrogel core 4054 from escaping. Each of the plurality of small openings 4078 has an average diameter of about 10 micrometers in one embodiment. Other dimensions of the small openings 4078 are acceptable as well. In this regard, although the constraining jacket 4058 has been described as having a woven configuration, any other configuration having a semi-permeable or porous attribute can be used. Finally, the constraining jacket 4058 material preferably allows for tissue in-growth and is textured to provide a grip or purchase within a disc space.

As indicated above, the hydrogel core 4054 is configured to expand from the dehydrated shape, shown in FIG. 67A, to a hydrated shape, shown in FIG. 68, following implantation. Manufacture of the hydrogel core 4054 is described in greater detail below. Generally speaking, the hydrogel core 4054 is constructed such that the hydrated shape is different from the dehydrated shape. In other words, the hydrogel core 4054 has a streamlined shape in the dehydrated state to facilitate implant, and preferably has a shape generally corresponding to the shape of a portion of a nucleus cavity (not shown) in the hydrated state. One example of the hydrated replacement disc nucleus 4050 is shown in FIG. 68. In the hydrated state, the hydrogel core 4054, and thus the replacement disc nucleus 4050, defines an anterior face 4082 (partially hidden in FIG. 68), a posterior face 4086, and opposing end plate faces 4090, 4094 (partially hidden in FIG. 68). The opposing end plate faces 4090, 4094 may also be referred to as a superior face and an inferior face, respectively. For the purposes of this disclosure, directional terminology such as “anterior,” “posterior,” “superior,” and “inferior” may be with reference with one possible orientation of the replacement disc nucleus 4050 within a nucleus cavity. Also, the terms “posterior” and “posteriorly” are used in their ordinary sense (i.e., from or through the rear-facing side of the patient) and are broad terms and they include an approach along any line generally behind and between the two lateral sides of the patient. It should be understood, however, that due to its unique sizing, the replacement disc nucleus 4050 can be orientated in any direction relative to a nucleus cavity or the world in general. As such, the directional terms are provided for purposes of illustration only, and should not be interpreted as limitations. As a point of reference, FIG. 68 again identifies the leading end 4066 and the trailing end 4074.

A comparison of the replacement disc nucleus 4050 in the dehydrated state (FIG. 67A) with that in the hydrated state (FIG. 68) illustrates the preferred transition in shape of the hydrogel core 4054. The hydrogel core 4054 has transitioned, upon hydration, from the streamlined configuration of FIG. 67 to a rectangular configuration of FIG. 68. In particular, in one embodiment, the hydrogel core 4054 in the hydrated state does not taper from the central portion 4070 to the leading end 4066 or the trailing end 4074. Instead, the hydrogel core 4054 has a relatively uniform height (z-axis in FIG. 68). In other words, with hydration, the hydrogel core 4054 transitions from a substantially convexo-convex cross-sectional shape to a rectangular (or plano-plano) shape. Further, in the hydrated state, the central portion 4070 of the hydrogel core 4054 is no longer curved along its length. As described in greater detail below, the replacement disc nucleus 4050 in the hydrated state generally adheres to the spacing requirements of a particular disc space.

This replacement disc nucleus 4050 preferably provides at least one of the following benefits: (a) restores and maintains the height of the damaged disc space; (b) restores and tightens the natural annulus to stop further degeneration and permit its healing; (c) restores the normal load-unload cycling and thus flushes out toxic by-products, bringing in fresh nutrients to the disc space; (d) allows a near-normal range of motion; and (e) relieves the movement-induced discogenic pain of the vertebral segment.

In addition, the replacement disc nucleus 4050 is advantageously insertable by way of a minimally invasive procedure as described herein. With reference to FIGS. 73-78, the replacement disc nucleus 4050 can be applied, to a patient by way of a minimally invasive access device which may be configured when inserted to provide greater access at a distal end thereof, e.g., near a surgical location near the spine. The term “access device” is used in its ordinary sense (i.e. a device that can provide access) and is a broad term and it includes structures having an elongated dimension and defining a passage, e.g., a cannula or a conduit. The increased access at the surgical location enables the surgeon to prepare the disc annulus through the access device and to insert the replacement disc nucleus 4050 into the intervetebral space through the access device. Thus, the minimally invasive apparatuses and methods enable the to surgeon to reduce the trauma cause by the procedure by which the replacement disc nucleus 4050 is inserted and to provide other benefits, such as reducing the length of recovery time.

Further details relating to this second replacement disc nucleus may be found in U.S. Pat. No. 6,602,291, issued Aug. 5, 2003, which is hereby incorporated herein by reference in its entirety.

3. Substantially Mushroom-Shaped Replacement Disc Nucleus

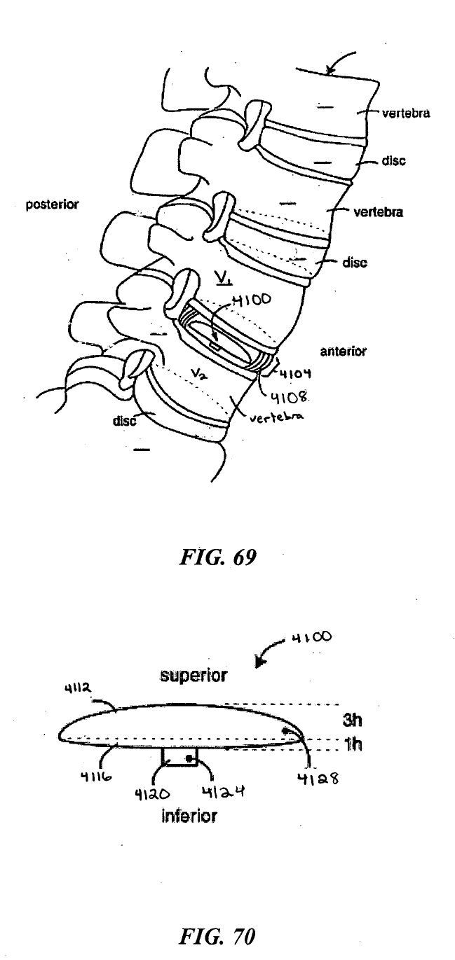

FIGS. 69 and 70 show another embodiment of a replacement disc nucleus 4100 that is substantially mushroom shaped. FIG. 69 illustrates one application of the replacement disc nucleus 4100. The natural disc 4104, which is located between the vertebrae V₁ and V₂, as shown in FIG. 69, is degenerated. The replacement disc nucleus 4100 is surgically embedded in the inter-vertebral space between vertebrae V₁ and V₂, and inside an annulus fibrosus 4108, as discussed in greater detail below in connection with FIGS. 73-77.

The replacement disc nucleus 4100 may comprise a solid polymer flattened into an oval disk. In general, any solid biocompatible material can be used, including various polymers and plastics, titanium, stainless steel, tantalum, chrome cobalt alloys, etc. Ultra-high molecular-weight polyethylene is presently preferred so that metal radiograph markers may be strategically placed in the replacement disc nucleus 4100.

As shown in FIG. 70, the replacement disc nucleus 4100 has a top half 4112 that is domed and has a crest that is about three times higher (“3h”) than the crest (“1h”) on a domed bottom half 4116. The replacement disc nucleus 4100 resembles a partially collapsed ellipsoid. Both top and bottom surfaces preferably are convex. The outside diameter of the replacement disc nucleus 4100 can vary, e.g., in the range of twenty to thirty-six millimeters. The overall height can also vary, e.g., in the range of eight to sixteen millimeters. The actual dimensions required depend on the size of the patient and the exact site to receive the replacement disc nucleus 4100. Such required sizes are discemable from patient radiographs, CT-scans, and MRI-scans.

In one embodiment, a peg 4120 extends down from the middle of the bottom-domed surface 4116. The peg 4120 is typically two to four millimeters long and is used to pin the replacement disc nucleus 4100 to the lower vertebrae, e.g., vertebrae V₂ in. FIG. 69. A pair of metal radiograph markers 4124 and 4128, e.g., one in the peg 4120 and one on an outside edge, are placed so that radiographs can be used to determine the replacement disc nucleus's in situ position. The replacement disc nucleus 4100 is surgically implanted into the hollowed out intervertebral space through a flap cut in the natural annulus fibrosus. Such “hollowing out” is commonly called a discectomy. The lower vertebra V₂ is prepared to receive the peg 4120 by clearing the material covering the top of the bone matrix. Bone cement is used around the peg 4120 to ensure a tight fit and immobile attachment of the disc to the lower vertebrae V2.

In one embodiment, the material making up the replacement disc nucleus 4100 is selected from a group of biocompatible, rigid or semi-rigid materials, including: titanium, stainless steel, surgical alloys, molybdenum alloys, cobalt chromium alloy, non-absorbable polymers, etc. Some embodiments of the replacement disc nucleus 4100 mimic the natural load-relieving, compressive functionality of a natural nucleus pulposus, while other embodiments do not. Further details relating to this replacement disc nucleus may be found in U.S. Pat. No. 6,146,422, issued Nov. 14, 2000, which is hereby incorporated herein by reference in its entirety.

4. Injectable Spinal Replacement disc nucleus

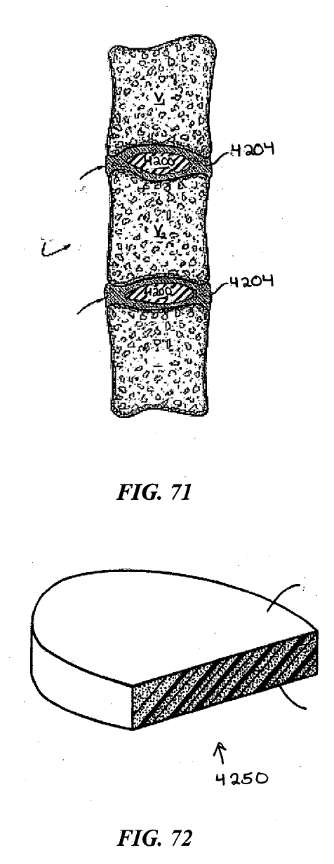

FIG. 71 shows another embodiment of a replacement disc nucleus 4200 applied to a segment of a patient's vertebral column. As shown, the replacement disc nucleus 4200 is interposed between adjacent ones of the individual vertebrae V₁ and V₂. The replacement disc nucleus 4200 is surrounded by the fibrillar outer annulus fibrosus 4204 of the patient's natural vertebral disc following removal of the gelatinous nucleus pulposus. The fibrillar outer annulus 4204 thus bounds and defines an inner cavity into which the replacement disc nucleus 4200 is injected in situ. This replacement disc nucleus 4200 may comprise a number of injectable materials, including hydrogels, thermoplastic elastomers, or a proteinaceous biopolymer, which thus fill the void space left following removal of the natural nucleus pulposus of the patient's natural vertebral disc. The replacement disc nucleus 4200 thus acts as a shock-absorber of sorts similar to the natural functions attributable to the removed gelatinous core.

In one embodiment, a biologically inert curable thermoplastic is injected through the annulus fibrosus and allowed to cure within the patient, until it has achieved a viscosity and hardness sufficient to support normal postural compressive loads. In another embodiment, virtually any suitable proteinaceous biopolymer may be used. In this regard, the term “proteinaceous biopolymer” and like terms mean a polymeric or copolymeric material which contains one or more units in the polymer chain comprised of natural, synthetic or sequence-modified proteins or polypeptides, and mixtures and blends of such polymeric and/or copolymeric materials.

One preferred biopolymer that may be used is a cross-linked reaction product of a two part mixture initially comprised of:

Part A: a water-soluble proteinaceous material of about 27-53% by weight of the mixture, and

Part B: di- or polyaldehydes present in a weight ratio of one part by weight to every 20-60 parts of protein present by weight in the mixture and water, optionally containing non-essential ingredients to make up the balance of the composition.

Part A of the mixture is preferably substantially an aqueous solution of a proteinaceous material of human or animal origin. Albumins including ovalbumins are preferred proteins, and serum albumins of human or animal origin are particularly preferred. The proteinaceous material may be a purified protein or a mixture in which the proteins such as serum albumins are the predominant ingredients. For example, the solid mixtures obtained by dehydration of blood plasma or serum, or of commercial solutions of stabilized plasma proteins, can be used to prepare Part A. These mixtures, generally referred to as plasma solids or serum solids, are known to contain albumins as their major ingredients, of the order of 50-90%. As used herein, the term “plasma” refers to whole blood from which the corpuscles have been removed by centrifugation. The term “serum” refers to plasma which has additionally been treated to prevent agglutination by removal of its fibrinogen and/or fibrin, or by inhibiting the fibrin clot formation through addition of reagents, such as citrate or EDTA. The proteinaceous material may also contain an effective amount of hemoglobin.

Part B may substantially be an aqueous solution of di- or polyaldehydes. A wide range of these substances exist, and their usefulness is restricted largely by availability and by their solubility in water. For example, aqueous glyoxal (ethandial) is useful, as is aqueous glutaraldehyde (pentandial). Water soluble mixtures of di- and polyaldehydes prepared by oxidative cleavage of appropriate carbohydrates with periodate, ozone or the like are also useful. Glutaraldehyde is the preferred dialdehyde ingredient of Part B. When Parts A and B are brought together, the resultant product rapidly hardens to a strong, flexible, leathery or rubbery material within a short time of mixing, generally on the order of 15-30 seconds. One material that may be used in this embodiment is commercially available from CryoLife, Inc. of Kennesaw, Ga. under the registered trademark “BIOGLUE”. See also, U.S. Pat. No. 5,385,606, which is hereby incorporated by references herein in its entirety.

The two components A and B noted above are either premixed and then applied, or simultaneously mixed and delivered through an in-line mixing/dispensing tip during the filling of the tissue-defined cavity. Upon reaction of the two components, the resulting biomaterial is a hydrogen that adheres to the surrounding tissue, intercalates into the voids of the surrounding tissues, is space filling, and is mechanically and biologically stable for some time. The material may be solid or sponge-like in appearance. Furthermore, it may contain organic or inorganic salts or other particulate matter to modify the physical properties of the resulting bioprosthetic device. Further details of the replacement disc nucleus 4200 may be found in U.S. patent application Ser. No. 09/983,537, filed on Oct. 24, 2001, which has been published as U.S. Publication No. 2002/0049498, and U.S. patent application Ser. No. 09/908,056 filed on Jul. 18, 2001, which are hereby incorporated herein by reference in their entirety.

5. Disc-Like Replacement Disc Nucleus

FIG. 72 shows another embodiment of a replacement disc nucleus 4150. In this embodiment, material forms a disc approximately the size of a natural, biological nucleus pulposus. This disc-like structure, which comprises the replacement disc nucleus 4150, is configured to be inserted into the patient's spine. Many different materials may be used. In one embodiment, hybrid materials used to induce and/or guide reformation of intervertebral disc tissue comprise biodegradable substrates that make up the disc. Biodegradable means that the substrate degrades into natural, biocompatible byproducts over time until the substrate is substantially eliminated from the implantation site and, ultimately, the body. In one embodiment, the rate of biodegradation of the substrate is preferably less than or equal to the rate of intervertebral disc tissue formation, such that the rate of tissue formation is sufficient to replace the support material that has biodegraded.

Further details relating to this embodiment of the replacement disc nucleus 4150 may be found in U.S. Pat. No. 6,240,926, issued Jun. 5, 2001, which is hereby incorporated herein by reference in its entirety and in U.S. patent application Ser. No. 10/167,503 filed on Jun. 13, 2002, which also is hereby incorporated by reference in its entirety.

C. Further Methods of Applying a Replacement Disc Nucleus

FIGS. 73-78 more particularly illustrate methods whereby a variety of embodiments of replacement disc nuclei, collectively referred to as a replacement disc nucleus 4300, may be delivered through an access device 4304 and implanted in an intervertebral space I defined between a first vertebra V₁ and a second vertebra V₂ and within an annulus fibrosus A. The replacement disc nucleus 4300 may be any suitable replacement disc nucleus, e.g., any of the replacement disc nucleuses 4000, 4050, 4100, 4150, 4200, or any other suitable replacement disc nucleus. Some methods or techniques of implanting the replacement disc nucleus 4300 may be similar to the methods described above in connection with FIG. 51 for implanting the fusion implant 2010.

In one method, access to the intervertebral space I is provided by inserting a retractor or access device 4304 into the patient. The access device 4304 may be configured in a manner similar to the expandable conduit 20 and may be inserted in a similar manner, e.g., over a dilator. The access device 4304 preferably has an elongate body 4308 that has a proximal end 4312 and a distal end 4316. The elongate body 4308 has a length between the proximal end 4312 and the distal end 4316 that is selected such that when the access device 4304 is applied to a patient during a surgical procedure, the distal end 4316 can be positioned inside the patient adjacent a spinal location, and, when so applied, the proximal end 4312 preferably is located adjacent the skin of the patient or outside the patient at a suitable height.

In one embodiment, the elongate body 4308 comprises a proximal portion 4320 and a distal portion 4324. The proximal portion 4320 may have a generally oblong or oval shape cross-section, a generally circular shape cross-section, or any other suitable shaped cross-section. The term “oblong” is used in its ordinary sense (i.e. having an elongated form) and is a broad term and it includes a structure having a dimension, especially one of two perpendicular dimensions, such as, for example, width or length, that is greater than another. The term “oval” is used in its ordinary sense (i.e., egg like or elliptical) and is a broad term and includes oblong shapes having curved portions and oblong shapes having parallel sides and curved portions. The distal portion 4324 preferably is expandable, as discussed above in connection with the expandable conduit 20, to the configuration illustrated in FIGS. 73-78. At least one passage 4328 extends through the elongate body 4308 between the proximal end 4312 and the distal end 4316. Further details of various additional embodiments of the access device 1504 may be found in U.S. patent application Ser. No. 10/678,744, filed Oct. 2, 2003, entitled MINIMALLY INVASIVE ACCESS DEVICE AND METHOD, which is hereby incorporated by reference herein in its entirety.

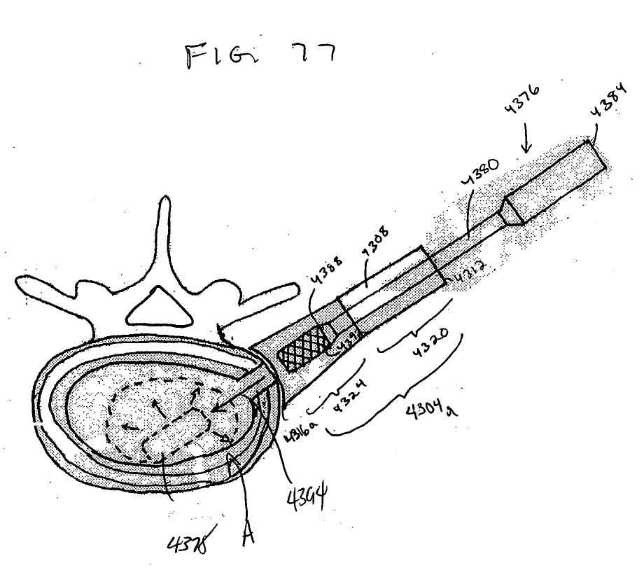

FIG. 75 shows that the access device 4304 is configured to be coupled with a viewing element 4332 in one embodiment. The distal portion 4324 of the access device 4304 has an aperture 4336 into which the viewing element 4332 can be inserted, such that a proximal portion of the viewing element 4332 lies external to the proximal portion 4320, and a distal portion of the viewing element 4332 lies within the distal portion 4324 of the access device 4304. In another embodiment, the viewing element 4332 may extend within the access device 4304 substantially entirely the length of the passage 4328. In other embodiments, the viewing element 4332 may be moved to the surgical location entirely externally to the access device 4304. The viewing element 4332 may be further configured to be removed from the access device 4304 during the procedure, as required.

The viewing element 4332 may be any suitable viewing element or portion thereof, such as an endoscope, a camera, loupes, a microscope, a lighting element, or a combination of the foregoing. The viewing element 4332 may be an endoscope, such as the endoscope 500, and a camera, which capture images to be displayed on a monitor, as discussed above.

The access device may be inserted generally posteriorly. Also, the terms “posterior” and “posteriorly” are used in their ordinary sense (i.e., from or through the rear-facing side of the patient) and are broad terms and they include an approach along any line generally behind and between the two lateral sides of the patient. In the illustrated methods, the distal end 4316 of the access device 4304 is inserted postero-laterally, to a surgical location adjacent to at least one vertebra and preferably adjacent to two vertebrae, e.g., the first vertebra V₁ and the second vertebra V₂, to provide access to at least a portion of the intervertebral space I. This approach is illustrated in the solid-line schematic representation of the access device 4304. In different methods, the access device 4304 may be inserted from a variety of different angles, e.g., posteriorly from directly between adjacent transverse processes to the more postero-lateral approach of FIGS. 73-78. These other example approaches are shown by the dashed line schematic representation of the access device in FIG. 73. In other methods, the access device 4304 may be inserted laterally, anteriorly, or from other approaches to provide access to at least a portion of the interbody space I. As discussed above, the access device 4304 can have a first configuration for insertion to the surgical location over the interbody space I and a second configuration wherein increased access is provided to the interbody space I. FIGS. 73-78 show that the second configuration may provide a cross-sectional area at the distal end 4316 that is larger than that of the first configuration at the distal end 4316. The distal portion 4324 of the access device 4304 may be expanded from the first configuration to the second configuration, as discussed above in connection 24, using the expander apparatus 200. When so expanded, the distal portion 4324, at the distal end 4316, defines a surgical space that exposes a portion of an external surface of an annulus A.

As discussed above, in one embodiment, the access device 4304 has a substantially circular cross-sectional shape in the proximal portion 4320. The access device 4304 may further have a circular cross-section near the proximal end 4312, near the distal end 4316, at the proximal and distal ends 4312, 4316, and from the proximal end 4312 to the distal end 4316. As discussed above, in another embodiment, the access device 4304 has an oblong cross-sectional shape in the proximal portion 4320. In particular, the access device 4304 may have an oblong cross-section near the proximal end 4312, near the distal end 4316, at the proximal and distal ends 4312, 4316, and from the proximal end 4312 to the distal end 4316.

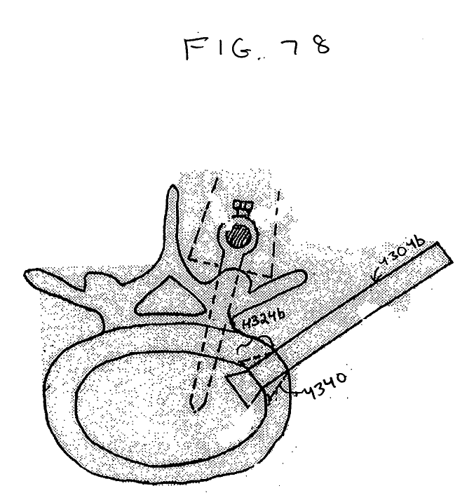

FIG. 76 and 77 show that an access device 4304a may also be provided that has a distal end 4316a shaped to follow a contour of the patient's anatomy. In one embodiment, the distal end 4316a of an access device 4304 is partially concave to complement the convex shape of the patient's intervertebral space I. When the concave distal end 4316a is placed adjacent the convex vertebral surface, the concave distal end 4316a of the access device 4304a more completely seals off the surgical location from other tissues, and provides a more defined surgical location than is possible with a flat distal end. In other embodiments, the distal portion 4324 of an access device 4304 may be otherwise shaped or configured to more closely contour the anatomy to which it will provide access. These configurations may provide increased, or more precise access to certain anatomical spaces.

FIG. 78 shows that an access device 4304b may be further configured such that a distal portion 4324b of the access device 4304b can be advanced into an aperture 4340 in the annulus fibrosus A. It may be configured such that the distal portion 4324b lies at least partially within the aperture 4340 in the tissue defining the annulus fibrosus A, or such that the distal portion 4324b extends beyond the aperture 4340 in the annulus fibrosus A into the intervertebral space I. In one embodiment, the transverse size, e.g., diameter, of the access device 4304b may be made substantially smaller than the transverse size, e.g., diameter, of the access device 4304 or the access device 4304a. The smaller diameter of the access device 4304b may provide a closer connection with the intervertebral space I that defines the surgical location, or enlarge an annulotomy (a hole in the annulus fibrosus A), depending on where the distal portion 4324b is expanded. According to one method of enlarging an annulotomy, the distal portion 4324b of the access device 4304b is sized to fit within an apertur & in the annulus fibrosus A in a first configuration, but enlarges the aperture when actuated to a second configuration (illustrated by the dashed lines in FIG. 78). Another advantage of the enlargement of the distal portion 4324b is that contact of the distal portion 4324b with the annulus fibrosus A causes the access device 4304b to be tethered to the disc so that movement with respect to the disc can be kept at a minimum. In other applications, the distal portion 4324b is expanded to engage the annulus fibrosus A to limit movement of the access device 4304b but not so much as to enlarge the annulus fibrosus A significantly.

In some methods of applying the replacement disc nucleus 4300, a second access device, such as an expandable conduit 20 or other suitable access device, may be inserted into the patient. For example, a second access device could be inserted through a postero-lateral approach on the opposite side of the spine, as indicated by an arrow 4348 on FIG. 73, to provide access to at least a portion of an intervertebral space, e.g., the intervertebral space I, on the contralateral side of the spine. Where provided, the second access device may provide access to the interbody space I at about the same time as the first access device 4304 or during a later or earlier portion of a procedure. In one method, the intervertebral space I is prepared to receive the replacement disc nucleus 4300 through a first access device, and the replacement disc nucleus 4300 is inserted from the other 'side of the spine using a second access device. In various applications, one or more replacement disc nuclei 4300 may be delivered through one or more access devices, such as the access device 4304, from different approaches. Any combination of single, multiple replacement disc nuclei, or replacement disc nucleus sub-components may be delivered through one or more access devices from any combination of one or more approaches, such as the approaches shown in FIG. 73, or any other suitable approach.

FIG. 74 shows a lateral view of a portion of a spine of a patient with the access device 4304 delivered thereto prior to treatment of the patient's natural disc. Advantageously, the access device 4304 may be configured so that when in the expanded configuration, the distal end 4316 does not extend beyond the locations of a nerve root 4352 or the spinal cord. The nerve root 4352 and the spinal cord are located outside the surgical space defined generally within the perimeter of the distal end 4316, and therefore are shielded from any implement or replacement disc nucleus or portion thereof delivered to the surgical location through the access device 4304. When in position, in addition to providing access to the interbody space I and the disc material therein, the distal portion 4324 may retract and shield the nerve root 4352 and spinal cord, and thereby protect the nerve root 4352 and spinal cord. The term “shield” as used in this context refers to the distal end 4316 of the access device 4304 being located between the surgical space and the nerve root 4352 or,the spinal cord, or in contact with the nerve root 4352 or the spinal cord without applying significant force, e.g., tension or displacement force, to the nerve root 4352 or the spinal cord.

As shown in FIG. 75, in some methods, suitable procedures may be performed to prepare the intervertebral space I to receive a replacement disc nucleus, e.g., the replacement disc nucleus 4300. First, a procedure may be performed whereby an aperture in the annulus fibrosus A is formed, e.g., an annulotomy procedure, through the access device 4304. Such a procedure may necessitate the deployment of additional surgical tools through the access device 4304. For example, an annulotomy may be performed using a trephine, and/or a knife, and/or one or more kerrisons. Other cutting instruments as well as non-cutting instruments may also be used to perform the annulotomy, e.g., lasers, RF, and other means well known to those of skill in the art. The aperture formed by these procedures provides access to the intervetebral space I beyond the annulus fibrosus A.

Once access to the intervertebral space I beyond the annulus fibrosus A has been provided, a disc evacuation tool 4356 may be inserted through the access device 4304 and used to remove at least a portion of the natural nucleus pulposus, and other disc material, as needed, through the access device 4304. The disc evacuation tool 4356 may comprise a shaver blade, RF device, laser, water jet or any other suitable instrument (e.g., a rongeur). Additional surgical tools may also be deployed through the access device 4304 as needed. Tools used in connection with the access device 4304 or other access devices described herein, such as the disc evacuation toot 4356, preferably are generally elongated such that when the tools are applied to a patient during a surgical procedure through the access device 4304, a distal portion of the tool can be positioned through the aperture in the annulus fibrosus A, into the intervertebral space I. When so applied, a proximal portion of the tools preferably extends proximally of the proximal end 4312 of the access device 4304.

In some methods, all of the natural nucleus material is removed, e.g., where it will serve no further purpose or will detract from the performance of the replacement disc nucleus 4300. In other methods, there may be no need to perform an annulotomy or to remove pre-existing nucleus pulposus. For example, disc degeneration may have produced a hole in the annulus fibrosus A through which the natural nucleus has been ejected, e.g., a disc herniation. Any of the foregoing procedures to prepare the intervertebral space I may be performed though the access device 4304, inserted as shown, or through any other access device described herein or through a second access device described herein which has been inserted through any suitable approach.

FIGS. 76-77 illustrate methods of applying replacement disc nuclei 4300 through the access device 4304. In particular, in FIG. 76, after the access device 4304 is actuated to the expanded configuration, a disc nucleus 4300 that is at least partially injectable is delivered through the access device 4304, through an aperture in the annulus fibrosus A and into the intervertebral space I. Disc nuclei that are at least partially injectable are illustrated in connection with FIGS. 66 and 71. In one application, though not shown in this FIG. 76, in order to facilitate insertion of the replacement disc nucleus 4300, visualization of the surgical location may be achieved in any suitable manner, e.g., by use of a viewing element 4332, as discussed above.

In one procedure, a passage or conduit 4360 may be inserted through the access device 4304, through the aperture in the annulus fibrosus A and into the intervertebral space I. The conduit 4360 preferably has a hollow, elongate body that extends between a proximal end 4364 and a distal end 4368. The length of the elongate body is selected such that when the passage 4360 is applied to a patient during a surgical procedure, the distal end 4368 can be positioned through the aperture in the annulus fibrosus A, into the intervertebral space I, and, when so applied, the proximal end 4364 extends proximally to the proximal end 4312 of the access device 4304. In one embodiment, the passage or conduit 4356 facilitates the delivery of at least a portion of the disc nucleus 4300, e.g., a filler material or medium, as discussed above, into the intervertebral space I. In one embodiment, the filler material is injected into a pliable enclosure, as discussed above.

As shown in FIG. 76, a container 4372 having the filler material of the injectable replacement disc nucleus 4300 remains external to the patient's body. The container may be attached to the proximal end 4364 of the passage 4360. As described above, the disc nucleus medium may comprise hydrogels, thermoplastic elastomers, proteinaceous biopolymers or other injectable materials. In one embodiment, the container 4372 may facilitate the injection of the disc nucleus medium through the passage 4360, through the aperture in the annulus A, into the intervertebral space I. In other embodiments, the injected medium may be pressurized to fill the intervertebral space I or to increase the disc height or volume. Different containers may be used, including a syringe.

In another application, the injectable replacement disc nucleus 4300 may be deployed by delivering the container 4372 through the access device 4304 into the intervertebral space I and then expelling its contents.

FIG. 77 illustrates further methods of delivering a replacement disc nucleus postero-laterally through the access device 4304a and through an aperture in the annulus fibrosus A into the intervertebral space I. In some applications, in order to facilitate insertion of the replacement disc nucleus 4300, visualization of the surgical location may be achieved in any suitable manner, e.g., by use of a viewing element 4332, as discussed above.

In one procedure, a gripping apparatus 4376 is coupled with one or more portions and/or surfaces of a replacement disc nucleus 4378 to facilitate insertion of the replacement disc nucleus. The gripping apparatus 4376 may be used in connection with replacement disc nuclei having solid form, e.g., as illustrated in connection with FIGS. 67A-70 and FIG. 72. In one embodiment, the gripping apparatus 4376 is similar to the tool 2032, described above. The gripping apparatus 4376 has an elongate body 4380 that extends between a proximal end 4384 and a distal end 4388. The length of the elongate body 4372 is selected such that when the gripping apparatus 4376 is inserted through the access device 4304a to intervertebral space I, the proximal end 4384 extends proximally of the proximal end 4312 of the access device 4304a. This arrangement permits the surgeon to manipulate the gripping apparatus 4376 proximally of the access device 4304a. The gripping apparatus 4376 has a grip portion 4392 that is configured to engage the replacement disc nucleus 4378.

In one embodiment, the grip portion 4392 comprises a clamping portion configured to firmly grasp opposing sides of the replacement disc nucleus 4378. The clamping portion may further comprise a release mechanism, which may be disposed at the proximal end 4384 of the gripping apparatus 4376, to loosen the clamping portion so that the replacement disc nucleus 4378 may be released once delivered to the intervertebral space I. In another embodiment, the grip portion 4392 comprises ajaw portion, such that a portion of the replacement disc nucleus 4378 fits within the jaw portion. In another embodiment, the grip portion 4392 comprises a malleable material that can conform to the shape of the replacement disc nucleus 4378 and thereby engage it. Other means of coupling the gripping apparatus 4376 to the replacement disc nucleus 4378 known to those of skill in the art could also be used, if configured to be inserted through the access device 4304a.

The replacement disc nucleus 4378 may be configured to be engaged by the grip portion 4392 of the gripping apparatus 4376. For example, the replacement disc nucleus 4378 could include a tab configured to be engaged by the grip portion 4392 of the gripping apparatus 4376. In one embodiment, the replacement disc nucleus 4378 is configured to fit within a jaw portion. In another embodiment, the replacement disc nucleus 4378 may be configured to fit within a clamping portion. In another embodiment, the replacement disc nucleus 4378 may be configured to mate closely with a corresponding surface in the grip portion 4392 of the gripping apparatus 4376.

In one method of delivering the replacement disc nucleus 4378 to the intervertebral space I, the gripping apparatus 4376 is coupled with the replacement disc nucleus 4378, as described above. The gripping apparatus 4376 and the replacement disc nucleus 4378 are advanced into the proximal end 4316 of the access device 4304, through the hole in the annulus fibrosus A, and further into the intervertebral space I, as indicated by an arrow 4394.

Once inserted, in some embodiments, the replacement disc nucleus 4378 may expand or swell to substantially fill the intervertebral space I e.g., in a manner similar to the replacement disc nucleus 4050, discussed above. In some procedures, the expansion or swelling of the disc nucleus 4378 may be encouraged or provided by a body fluid that hydrates and thereby enlarges the disc nucleus 4378 in situ. In other procedures, the disc nucleus 4378 is self-expanding to substantially fill the intervertebral space I. In still other procedures, external means may be used to expand or enlarge the disc nucleus 4378. These external means may include a device for injecting a liquid upon or within the disc nucleus 4378, e.g., a syringe, the passage 4356 and container 4368 combination described above, or other means well known to those of skill in the art that might pass material through the access device 4300, through the aperture in the annulus fibrosus A, and into the intervertebral space I.

Although not shown in either FIG. 76 or 77, any apertures formed in the annulus fibrosus A may be closed to prevent or minimize the escape or herniation of the replacement disc nuclei 4300, 4378, or similar replacement disc nuclei, or any portion thereof. Such a procedure may necessitate the deployment of additional surgical tools through any of the access devices described herein. For example, tools may be provided to stitch, suture, tape, plug or fill the void (e.g., to deliver a hydrogel plug), or otherwise repair the aperture in the annulus fibrosus A, either permanently or temporarily, may optionally be deployed. In some embodiments, artificial annulus fibrosus may be inserted through any of the access devices described herein and surgically attached to the natural annulus A to strengthen and reinforce the tissue.

Although the forgoing procedures are described in connection with a single level postero-lateral procedure, other procedures are possible. For example, multiple level nucleus replacement could be performed with one or more expandable conduit 20 or other suitable access device. As discussed above, other applications are also possible in which the access device 4304 is not expanded prior to delivery of the replacement disc nuclei 4300, 4378 or other similar nuclei. In such applications, the access device 4304 remains in the first configuration while the steps described above are performed, or a non-expandable access device may be provided. Also, other approaches could be adopted,. e.g., anterior, posterior, transforaminal, or any other suitable approach. In one application, a replacement disc nucleus 4300, 4378 is inserted at the L5-S1 disc or at the L5-L4 disc anteriorly through the access device 4304.

As shown in FIG. 78, a nucleus replacement procedure could also be combined with the insertion of a stabilization device between two adjacent vertebrae. The stabilization device may comprise a rigid system immobilizing the vertebrae V₁ and V₂ relative to each other, or may preserve motion between the vertebrae by means of a more dynamic system. Moreover, the access devices described herein may be used to perform all of these procedures, using single or multiple insertions. In one embodiment, a single access device is used first to replace a nucleus pulposus in an intervertebral space I with a replacement disc nucleus, and then to deliver and configure a stabilization device to the two vertebrae defining the intervertebral space I.

Although the methods discussed above are particularly directed to the insertion of a replacement disc nucleus, the apparatuses and systems described herein may also be used advantageously to extract or remove the replacement disc nuclei described herein, in a process known as revision. In one application, the means by which the aperture in the annulus A is closed may be configured to facilitate future annulotomies. Furthermore, any of the replacement disc nuclei may be configured to facilitate subsequent removal thereof. The gripping apparatus 4376 may also be further configured to facilitate removal as well as insertion. By providing minimally invasive access to the interbody space I, the access devices described herein may be used analogously, as described above with reference to the removal of the natural nucleus pulposus, to remove a previously inserted replacement disc nucleus. In one application, the previously inserted replacement disc nucleus may then be replaced with a-new replacement disc nucleus through the access devices described herein.

The foregoing methods and apparatuses advantageously provide minimally invasive treatment of disc conditions in a manner that preserves some degree of motion between the vertebrae on either side of a replaced nucleus. Accordingly, trauma to the patient may be reduced thereby, and recovery time shortened. As discussed above, many of the replacement disc nucleuses provide a more normal post-recovery range of motion of the spine, which can reduce the need for additional procedures.

It will be understood that the foregoing is only illustrative of the principles of the invention, and that various modifications, alterations, and combinations can be made by those skilled in the art without departing from the scope and spirit of the invention.

1. A method of replacing a portion of a disc of a patient, the disc having an annulus and a nucleus, comprising:
inserting an access device through an incision in the skin of the patient generally postero-laterally and advancing the access device until a distal portion thereof is located adjacent-the spine, said access device being inserted in a first configuration having a first cross-sectional area at the distal portion thereof;
configuring said access device such that the distal portion thereof is enlarged from the first configuration to a second configuration wherein the distal portion extends across at least a portion of the disc;
advancing an annulotomy tool through the access device to the intervertebral space;
forming an aperture in the annulus;
advancing a disc evacuation tool through the access device and through the aperture;
removing at least a portion of the nucleus through the access device to at least partially evacuate the intervertebral space; and
delivering a replacement disc nucleus into the partially evacuated intervertebral space through the access device.

2. The method of claim 1, wherein the replacement disc nucleus comprises an injectable material.

3. The method of claim 2, wherein the injectable material is chosen from a group comprising: hydrogels, thermoplastic elastomers, and proteinaceous biopolymers.

4. The method of claim 1, wherein the replacement disc nucleus comprises an expandable element.

5. The method of claim 4, wherein the expandable element comprises:
a bag in a collapsed configuration, wherein the bag may be inflated or allowed to expand.

6. The method of claim 5, wherein the replacement disc nucleus further comprises a biocompatible material, which is injected into the bag in an expanded configuration.

7. The method of claim 5, wherein the bag can be inflated to an expanded configuration with a gas or liquid after insertion.

8. The method of claim 7, wherein a tool is inserted through the access device in order to inflate the bag.

9. The method of claim 5, wherein the bag comprises a self-expanding frame that assumes a collapsed state for insertion, and an expanded state once inserted.

10. The method of claim 9, wherein the self-expanding frame is composed of a shape-memory material.

11. The method of claim 6, wherein the biocompatible material includes tissues, cells, or extracellular matrix components.

12. The method of claim 6, wherein the biocompatible material includes autograft nucleus pulposus, allograft nucleus pulposus or xenograft nucleus pulposus.

13. The method of claim 6, wherein the biocompatible material includes morselized nucleus or annulus from the disc.

14. The method of claim 4, wherein the expandable element comprises:
a hydrogel core configured to expand from a dehydrated state to a hydrated state, the hydrogel core being configured to have a dehydrated shape in the dehydrated state that facilitates insertion of the replacement disc nucleus through an opening in an annulus fibrosus and being generally different from a hydrated shape of the hydrogel core in the hydrated state,
wherein the hydrogel core is surrounded by a constraining jacket, the constraining jacket being flexible but substantially inelastic.

15. The method of claim 14, wherein the hydrogel core comprises a keratin hydrogel.

16. The method of claim 14, wherein the constraining jacket is porous enough to allow the hydrogel core to interact with bodily fluids.

17. The method of claim 16, wherein the hydrogel core is dehydrated prior to insertion.

18. The method of claim 1, wherein the replacement disc nucleus comprises:
an ellipsoidal body having a convex top side for contracting and articulating with an end-plate cartilage of a top vertebrae and a convex bottom side for an immobile contact with a bottom vertebrae;
said convex top side having a dome -crest that exceeds a dome crest of said convex bottom side by a factor of approximately three; and
a peg extending from said bottom side of the ellipsoidal body and providing for a pinning action with respect to said bottom vertebrae.

19. The method of claim 1, wherein the replacement disc nucleus comprises disc cells and a biodegradable substrate.

20. The method of claim 19, wherein the biodegradable substrate is bioactive.

21. A method of treating the spine of a patient, comprising:
inserting an access device through a minimally invasive incision in the skin of the patient;
advancing the access device until a distal portion thereof is located adjacent the spine;
expanding said access device from a first configuration to a second configuration, the second configuration having an enlarged cross-sectional area at the distal portion thereof such that the distal portion extends across at least a portion of a disc;
delivering a replacement disc nucleus into an intervertebral space through the access device.

22. A device for providing access to a surgical location within a patient, said device comprising:
an elongate body having a proximal end, a distal end, and a passage extending therebetween, the elongate body defining a length between the proximal and distal ends such that the proximal end can be positioned outside the patient and the distal end can be positioned inside the patient adjacent the surgical location, the distal end being shaped to conform to a contour of an anatomical structure near the surgical location; and
wherein the elongate body is actuatable between a first configuration sized for insertion into the patient and a second configuration wherein the cross-sectional area of said passage at a first location is greater than the cross-sectional area of said passage at a second location, wherein the first location is distal to the second location.

23. A system for replacing a portion of a disc having a nucleus and an annulus, comprising
the access device of claim 22;
an annulotomy tool for forming an aperture in the annulus through the access device; and
a disc evacuation tool for removing at least a portion of the nucleus through the access device.

24. A device for accessing an intervertebral disc of a patient having a nucleus and an annulus, said device comprising:
an elongate body having a proximal end, a distal end, and a passage extending therebetween, the elongate body defining a length between the proximal and distal ends such that the proximal end can be positioned outside the patient and the distal end can be advanced inside the patient and into the annulus; and
wherein the elongate body is actuatable between a first configuration sized for advancement to spine and a second configuration wherein the cross-sectional area of said passage at a first location is greater than the cross-sectional area of said passage at a second location, wherein the first location is distal to the second location.

25. The device of claim 24, wherein the distal end can be further advanced through the annulus.

26. The device of claim 24, wherein the elongate body actuating between a first configuration and a second configuration enlarges a hole in the annulus.

27. A device for accessing an intervertebral disc of a patient having a nucleus and an annulus, said device comprising:
an elongate body having a proximal end, a distal end, a passage extending therebetween, and a viewing element aperture located near the distal end, the elongate body defining a length between the proximal and distal ends such when the distal end is advanced into the patient to the annulus, the proximal end is positioned outside the patient; and
a viewing element extending through the aperture into the passage.
